(19) United States
(12) Patent Application Publication
(10) Pub. No.: US 2008/0145372 A1
(43) Pub. Date: Jun. 19, 2008

(54) Bioreductively-Activated Prodrugs

(75) Inventors: Peter David Davis, Oxford (GB); Matthew Alexander Naylor, Middlesex (GB); Peter Thomson, Middlesex (GB); Steven Albert Everett, Middlesex (GB); Michael Richard Lacey Stratford, Middlesex (GB); Peter Wardman, Middlesex (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: ANGIOGENE, PHARMACEUTICALS LIMITED, Oxfordshire (GB); THE GRAY LABORATORY CANCER RESEARCH TRUST, Middlesex (GB)

(21) Appl. No.: 11/663,469

(22) PCT Filed: Sep. 26, 2005
(86) PCT No.: PCT/GB05/03697

§ 371 Date: May 29, 2007

(30) Foreign Application Priority Data
Sep. 24, 2004 (GB) 0421294.0

(51) Int. Cl. A61K 39/395; C07H 19/09; A61K 31/7068; A61P 35/00; A61K 31/7052; A61P 19/02; A61K 38/44

(52) U.S. Cl. 424/178.1; 536/28.51; 514/49; 536/27.4; 514/46; 424/94.4; 514/44

(57) ABSTRACT

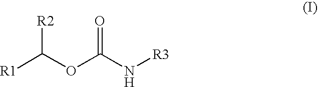

The present invention relates to a compound of formula (1), or a pharmaceutically acceptable salt thereof, Formula: (1); wherein: R1 is a substituted aryl or heteroaryl group bearing at least one nitro or azido group or is an optionally substituted benzoquinone, optionally substituted naphthoquinone or optionally substituted fused heterocycloquinone: R2 is H, optionally substituted alkyl, optionally substituted alkenyl, optionally substituted alkynyl, optionally substituted cycloalkyl, optionally substituted heterocycloalkyl, aryl or heteroaryl; and R3 is selected such that R3NH2 represents a cytotoxic nucleoside analogue or an ester or phosphate ester prodrug of a cytotoxic nucleoside analogue, with the proviso that if R1 is an aryl group then R2 is not H.

This invention relates to compounds useful in the treatment of cell proliferation disorders. More particularly the invention relates to a series of compounds that are activated under hypoxic conditions.

Many drugs used in conventional cancer chemotherapy are toxic to growing cancer cells but lack complete specificity. Thus other normal tissues are affected and ensuing side effects limit the dose that can be administered. Therefore the exposure of the cancerous tumour to the compound, and in turn the effectiveness of the therapy, is limited. There is a need for drugs that target the tumour more selectively.

Many solid tumours exhibit regions of hypoxia (low oxygen tension). Inadequate blood supply to the central regions of the tumour results in hypoxia that can be chronic or acute. This hypoxia represents a challenge to effective therapy by radiation or by conventional chemotherapy since hypoxic regions are often more resistant to these modalities. It has been suggested, however, that tumour hypoxia can be used to target tumours for drug action (Kennedy, Cancer Res. 1980, 40, 2356-2360.). One particular method of using the hypoxic regions of tumours for drug targeting is the selective activation of prodrugs under conditions of low oxygen tension. A concept has been advanced whereby the activity of a cytotoxic compound can be masked by a trigger moiety which, under hypoxic conditions, mediates fragmentation of the masked cytotoxic compound into the active cytotoxic agent (Denny, Lancet Oncol 2000, 1, 25-9). Compounds attempting to utilize this concept typically consist of the trigger moiety attached, often via a linker moiety, to a cytotoxic moiety (the effector).

Hypoxia is also a feature of the rheumatoid arthritic joint (Rothschild Semin Arthritis Rheum 1982, 12, 11-31). Cell proliferation is also a feature of the arthritic joint. Systemic antiproliferative drugs (for example methotrexate) are used in the therapy of rheumatoid arthritis but are limited by side effects. Psoriatic lesions are also characterized by cell proliferation and hypoxia (Dvorak Int Arch Allergy Immunol. 1995, 107, 233-5.

A number of hypoxic trigger moieties have been disclosed including nitrobenzenes, nitronaphthalenes, nitroimidazoles, nitrofurans, nitrothiophenes, nitropyrroles, nitropyrazoles, benzoquinones, naphthoquinones, indoloquinones and azidobenzenes (for some examples see Naylor, Mini Rev. Med. Chem. 2001 1, 17-29; Tercel, J. Med. Chem. 2001, 44, 3511-3522 and Damen, Bioorg. Med. Chem. 2002, 10, 71-77).

A number of effector moieties have been utilised in the art including nitrogen mustards, phosphoramide mustards, taxanes, enediynes and indole derivatives (for some examples see Naylor, loc cit and Papot, Curr. Med. Chem. Anti Cancer Agents 2002, 2, 155-185).

Hypoxic triggers joined to effectors via a linking group have been described wherein the linking group consists of a carbamate. In these cases it is intended that the intermediate carbamic acid, formed by the initial hypoxia-driven fragmentation, further fragments to give the active agent. A number of related compounds have been synthesised as prodrugs for use in gene-directed enzyme prodrug therapy or antibody-directed enzyme prodrug therapy. These strategies have been disclosed in for example: Berry, J Chem Soc Perkin Trans. 1, 1997, 1147-56; Denny WO 00/64864; Mauger, J Med Chem 1994, 37, 3452-58; Hay, Bioorg Med Chem Lett 1995, 5, 2829; Tercel, Bioorg Med Chem Lett 1996, 6, 2741; Hay, Bioorg Med Chem Lett 1999, 9, 3417-22; Hay, Bioorg Med Chem Lett 1999, 9, 2237-42.

Despite a body of work regarding compounds that break down selectively under low oxygen tensions to release an anticancer agent, no such compound is yet in clinical use. A number of problems have been encountered in the development of such compounds. A lack of stability of the prodrugs towards non-bioreductive processes has been regularly encountered. For example Sartorelli (J Med Chem 1986, 29, 84-89) has described a series of 5-fluorouracil prodrugs designed to fragment to give 5-fluorouracil under hypoxic conditions but these compounds did not prove useful in this respect due to chemical instability. Borch (J Med Chem 2000, 43, 3157-3167) has described a series of naphthoquinones designed to release phosphoramide mustards on quinone reduction but these compounds were unstable in cell cytotoxicity assays and released the active agent by mechanisms other than quinone reduction. Similarly the carbonate-linked taxol prodrugs described by Damen (loc cit) were reported to be unstable towards enzymatic hydrolysis in cellular assays, thereby releasing taxol by a non-reductive process. Borch (J Med Chem 2001, 44, 74-77) has also described a series of hypoxia activated nitroheterocyclic phosphoramidates which were unstable in vivo, displaying rapid metabolism and consequent elimination half-lives of only a few minutes. Wilson (J Med Chem 2001, 44, 3511-3522) has disclosed a series of nitroheteroaryl quaternary salts as bioreductive prodrugs of mechlorethamine but concluded that the compounds were too unstable with regard to non-specific release of mechlorethamine to be of use as bioreductive agents. Thus prodrugs showing improved stability towards non-reductive processes would have advantage.

A further consideration is the rate of release of the active drug under hypoxic conditions. To be effective the bioreductively activated prodrug needs to deliver the drug at a rate which competes with clearance of the prodrug and diffusion of the drug out of the solid tumour. Prodrugs that fragment faster than those in the art, or that fragment more efficiently at oxygen tensions commonly found in solid tumours, would be advantageous.

A number of cytotoxic nucleoside analogues are in clinical use, or have been the subject of clinical trials, as anticancer agents. Examples of such analogues include cytarabine, gemcitabine, troxacitabine, decitabine, tezacitabine, DMDC, cladribine, clofarabine, 5-azacytidine, 4′-thio-aracytidine, cyclopentenylcytosine and 1-(2-C-cyano-2-deoxy-β-D-arabino-pentofuranosyl)-cytosine. Another example of such a compound is fludarabine phosphate. These compounds are administered systemically and have side effects typical of cytotoxic agents with little or no specificity for tumour cells over proliferating normal cells.

A number of prodrugs of cytotoxic nucleoside analogues are reported in the art. Examples are N4-behenoyl-1-β-D-arabinofuranosylcytosine, N4-octadecyl-1-β-D-arabinofuranosylcytosine, N4-palmitoyl-1-(2-C-cyano-2-deoxy-β-D-arabino-pentofuranosyl)cytosine, P-4055 (cytarabine 5′-elaidic acid ester), and fludarabine phosphate. In general these prodrugs are converted into the active drugs mainly in the liver and systemic circulation and display little or no selective release of drug in the tumour tissue. Capecitabine, a prodrug of 5′-deoxy-5-fluorocytidine (and eventually of 5-fluorouracil), is metabolised both in the liver and in the tumour tissue. A series of capecitabine analogues containing “an easily hydrolysable radical under physiological conditions” has been claimed by Fujiu et al. (U.S. Pat. No. 4,966,891). The series described by Fujiu includes N4 alkyl and aralkyl carbamates of 5′-deoxy-5-fluorocytidine and the implication is that these compounds will be activated by hydrolysis under normal physiological conditions to provide 5′-deoxy-5-fluorocytidine. Despite the inclusion of compounds having a 4-nitrobenzylcarbamate substituent in the claims there was no suggestion of any compound that might act as a bioreductive prodrug. A series of cytarabine N4-carbamates has been by reported by Fadl et al (Pharmazie. 1995, 50, 382-7) in which compounds were designed to convert into cytarabine in the liver and plasma. Despite the fact that one compound described contained a 4-nitrobenzyl moiety, no suggestion was made that these compounds might be activated selectively within tumours. WO 2004/041203 discloses prodrugs of gemcitabine, some of which are N4-carbamates. These compounds were designed to overcome the gastrointestinal toxicity of gemcitabine and were intended to provide gemcitabine by hydrolytic release in the liver and plasma after absorption of the intact prodrug from the gastrointestinal tract. The generality of the application to human therapy of the compounds of WO 2004/041203 appears questionable as some examples showed efficient release only in mouse plasma and not in human plasma or liver preparations. No mention was made of any compounds that might selectively provide gemcitabine at the site of a solid tumour.

Nomura et al (Bioorg Med. Chem. 2003, 11, 2453-61) have described acetal derivatives of 1-(3-C-ethynyl-β-D-ribo-pentofaranosyl)cytosine which, on bioreduction, produced an intermediate that required further hydrolysis under acidic conditions to produce a cytotoxic nucleoside compound.

It is an object of this invention to provide novel prodrugs that on bioreductive activation break down to release a cytotoxic nucleoside analogue.

Thus according to one aspect of the invention we provide a compound of formula (1):

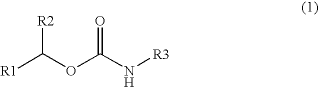

wherein:

R1 is a substituted aryl or heteroaryl group bearing at least one nitro or azido group or is an optionally substituted benzoquinone, optionally substituted naphthoquinone or optionally substituted fused heterocycloquinone;

R2 is H, optionally substituted alkyl, optionally substituted alkenyl, optionally substituted alkynyl, optionally substituted cycloalkyl, optionally substituted heterocycloalkyl, aryl or heteroaryl; and

R3 is selected such that R3NH₂ represents a cytotoxic nucleoside analogue or an ester or phosphate ester prodrug of a cytotoxic nucleoside analogue, with the proviso that if R1 is an aryl group then R2 is not H.

For the avoidance of doubt, the invention extends to the compounds of formula (1) in pharmaceutically acceptable salt form. Further, for the avoidance of doubt, the invention extends to the compounds of formula (1) in pharmaceutically acceptable solvate form.

In an embodiment of the invention, R3 is a group of formula (2), (3) or (4)

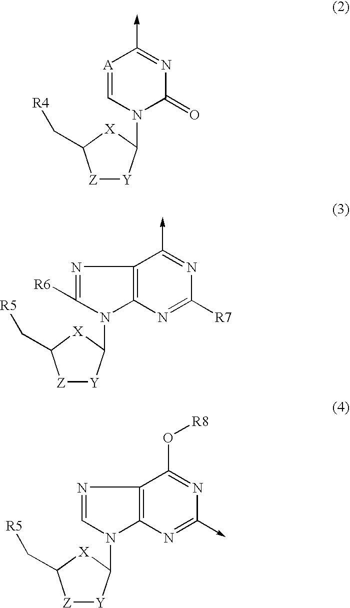

in which:

A is N, CF or CH;

X is O or S;

Y is CH₂, CHOH, CHO(CO)alkyl, CHF, CF₂, CHCN, C═CH₂, or C═CHF;

Z is CHOH, CR9′OH, CHOP(O)(OH)₂, CHOC(O)alkyl or 0;

R4 is H, OH, OP(O)(OH)₂ or OC(O)alkyl;

R5 is OH, OP(O)(OH)₂ or OC(O)alkyl;

R6 is H, Cl or F;

R7 is H, Cl or F;

R8 is H or alkyl; and

R9′ is alkyl, alkenyl or alkynyl, with the proviso that, in this embodiment, R3NH₂ does not represent the natural nucleosides cytidine, 2′-deoxycytidine, adenosine, 2′-deoxyadenosine, guanosine, 2′deoxyguanosine or a cytidine, 2′-deoxycytidine, adenosine, 2′-deoxyadenosine guanosine, 2′deoxyguanosine prodrug, further, typically when R4 is H then A is CF, X is O and Y is CHOH or CHO(CO)alkyl.

As used herein the term “alkyl”, alone or in combinations, means a straight or branched-chain alkyl group containing from one to seven, preferably a maximum of four, carbon atoms such as methyl, ethyl, propyl, isopropyl, butyl, sec-butyl, t-butyl and pentyl. Typically, an alkyl group or moiety is a linear or branched alkyl group or moiety containing from 1 to 6 carbon atoms such as a C₁-C₄ or C₁-C₂ alkyl group or moiety. More preferably, an alkyl group or moiety is methyl.

An alkenyl group may be for example an olefinic group containing from two to seven carbon atoms, for example ethenyl, n-propenyl, i-propenyl, n-butenyl, i-butenyl, s-butenyl and t-butenyl. Typically an alkenyl group is a C₂-C₆ alkenyl group, for example a C₂-C₄ alkenyl group. An alkenyl group typically contains only one double bond.

As used herein, an alkynyl group is a linear or branched alkynyl group. Typically an alkynyl group is a C₂-C₆, for example a C₂-C₄ alkynyl group, for example ethynyl, n-propynyl or n-butynyl. Typically, an alkynyl group contains only one triple bond. An alkynyl group may be for example an ethynyl, propynyl or butynyl group.

Optional substituents which may be present on alkyl, alkenyl or alkynyl groups include one or more substituents selected from halogen, amino, monoalkylamino, dialkylamino, hydroxy, alkoxy, alkylthio, alkylsulphonyl, aryl, heteroaryl, heterocycloalkyl, acylamino, alkoxycarbonylamino, alkanoyl, acyloxy, carboxy, sulphate or phosphate groups. Preferably, the alkyl, alkenyl or alkynyl groups are unsubstituted or substituted by 1, 2 or 3 substitutents. For the avoidance of doubt, these substituents are themselves unsubstituted.

Preferably, the substituents on an alkyl, alkenyl or alkynyl group or moiety are selected from halogen, amino, mono(C₁-C₄ alkyl)amino, di(C₁-C₄ alkyl)amino, hydroxy, C₁-C₄ alkoxy, C₁-C₄ alkylthio, (C₁-C₄ alkyl)sulphonyl groups, aryl, heteroaryl, heterocycloalkyl, acylamino, (C₁-C₄)alkoxycarbonylamino, (C₁-C₄)alkanoyl, acyloxy, carboxy, sulphate or phosphate groups. More preferably, the substituents on an alkyl, alkenyl or alkynyl group or moiety are selected from halogen, amino, mono(C₁-C₂ alkyl)amino, di(C₁-C₂ alkyl)amino or hydroxy. More preferably an alkyl, alkenyl or alkynyl group is unsubstituted.

The term “halogen” means fluorine, chlorine, bromine or iodine.

The term aryl means an unsubstituted phenyl group or a phenyl group carrying one or more, preferably one to three, substituents examples of which are halogen, optionally substituted alkyl, hydroxy, nitro, azido, cyano, amino, alkylamino, dialkylamino, acylamino, alkoxycarbonylamino, alkanoyl, acyloxy, carboxy, aminocarbonyl, alkylaminocarbonyl, dialkylaminocarbonyl, alkylthio and alkoxy. Typically, an aryl group is an unsubstituted phenyl group or a phenyl group substituted with 1, 2 or 3 unsubstituted substituents selected from halogen, C₁-C₆ alkyl, hydroxy, nitro, azido, cyano, amino, C₁-C₄ alkylamino, di(C₁-C₄)alkylamino, acylamino, C₁-C₄ alkoxycarbonylamino, C₁-C₄ alkanoyl, acyloxy, carboxy, aminocarbonyl, C₁-C₄ alkylaminocarbonyl, di(C₁-C₄)alkylaminocarbonyl, (C₁-C₄)alkylthio, C₁-C₄ alkoxy, C₁-C₄haloalkyl, and C₁-C₄ haloalkoxy.

Preferably an aryl group carries, where appropriate, in addition to the specified nitro or azido group, 0, 1, 2 or 3 unsubstituted substituents selected from halogen, C₁-C₆ alkyl, hydroxy, amino, C₁-C₄ alkylamino, di(C₁-C₄)alkylamino, carboxy, (C₁-C₄)alkylthio, C₁-C₄ alkoxy, C₁-C₄ haloalkyl, and C₁-C₄ haloalkoxy. More preferably, these preferred substituents are selected from halogen, C₁-C₂ alkyl, C₁-C₂ haloalkyl, C₁-C₂ alkoxy and C₁-C₂ haloalkoxy substituents.

As used herein, alkoxy is a said alkyl group which is attached to an oxygen atom.

As used herein, alkylthio (also known as a thioalkoxy) is a said alkyl group which is attached to a sulphur atom.

As used herein, a haloalkyl or haloalkoxy group is a said alkyl or alkoxy group, substituted by one or more said halogen atoms. Typically, a haloalkyl or haloalkoxy group is substituted by 1, 2 or 3 said halogen atoms. Preferred haloalkyl and haloalkoxy groups include perhaloalkyl and perhaloalkoxy groups such as —CQ₃ and —OCQ₃ wherein Q is said halogen atom, for example chlorine or fluorine. Particularly preferred haloalkyl groups are —CF₃ and —CCl₃. Particularly preferred haloalkoxy groups are —OCF₃ and —OCCl₃.

The term heteroaryl is defined herein as a monocyclic or fused bicyclic aromatic group containing one to four heteroatoms selected in any combination from N, S or O atoms. A heteroaryl group is typically a 5- to 10-membered ring, such as a 5- or 6-membered ring, containing at least one heteroatom, for example 1, 2, or 3 heteroatoms chosen from N, S or O atoms or a fused bicyclic group in which a 5- to 6-membered heteroaryl ring is fused to a phenyl ring, to a 5- or 6-membered heteroaryl ring, to a 5- to 6-membered non-aromatic, saturated or unsaturated heterocycloalkyl ring or to a C₅₋₆ cycloaliphatic carbocyclic ring. Preferably a heteroaryl group is monocyclic. Examples of heteroaryl groups include pyridyl, pyrimidyl, furyl, thienyl, pyrrolyl, pyrazolyl, indolyl, benzofuryl, benzothienyl, benzothiazolyl, oxazolyl, isoxazolyl, thiazolyl, isothiazolyl, imidazolyl, triazolyl, quinolyl and isoquinolyl groups. Preferred examples of heteroaryl groups include imidazolyl, thienyl, and furanyl groups.

A heteroaryl group can carry one or more, preferably one to three, substituents examples of which are halogen, optionally substituted alkyl, hydroxy, nitro, azido, cyano, amino, alkylamino, dialkylamino, acylamino, alkoxycarbonylamino, alkanoyl, acyloxy, carboxy, aminocarbonyl, alkylaminocarbonyl, dialkylaminocarbonyl, alkylthio and alkoxy. Typically, a heteroaryl group is an unsubstituted heteroaryl group or a heteroaryl group substituted with 1, 2 or 3 unsubstituted substituents selected from halogen, C₁-C₆ alkyl, hydroxy, nitro, azido, cyano, amino, C₁-C₄ alkylamino, di(C₁-C₄)alkylamino, acylamino, C₁-C₄ alkoxycarbonylamino, C₁-C₄ alkanoyl, acyloxy, carboxy, aminocarbonyl, C₁-C₄ alkylaminocarbonyl, di(C₁-C₄)alkylaminocarbonyl, (C₁-C₄)alkylthio, C₁-C₄ alkoxy, C₁-C₄ haloalkyl, and C₁-C₄ haloalkoxy.

Preferably a heteroaryl group carries, where appropriate, in addition to the specified nitro or azido group, 0, 1, 2 or 3 unsubstituted substituents selected from from halogen, C₁-C₆ alkyl, hydroxy, nitro, azido, cyano, amino, C₁-C₄haloalkyl, C₁-C₄ alkoxy and C₁-C₄ haloalkoxy. More preferably, these substituents are selected form from halogen, C₁-C₂ alkyl or C₁-C₂ haloalkyl.

A heterocycloalkyl ring is typically a non-aromatic, saturated or unsaturated C₃₋₁₀ carbocyclic ring in which one or more, for example, 1, 2 or 3, of the carbon atoms are replaced by a heteroatom selected from N, O or S. Preferably, a heterocycloalkyl ring is a 5- to 6-membered heterocycloalkyl ring. Saturated heterocycloalkyl groups are preferred. The term heterocycloalkyl ring includes heterocycloalkyl groups containing 3-6 carbon atoms and one or two oxygen, sulphur or nitrogen atoms. Particular examples of such groups include azetidinyl, pyrrolidinyl, piperidinyl, homopiperidinyl, piperazinyl, homopiperazinyl, morpholinyl or thiomorpholinyl groups.

Substituents which may be present on a heterocycloalkyl ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate.

Typically, a heterocycloalkyl ring is an unsubstituted heterocycloalkyl group or a heterocycloalkyl group substituted with 1, 2 or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, C₁-C₄ alkylamino, di(C₁-C₄)alkylamino, carboxy, (C₁-C₄)alkoxycarbonyl, aminocarbonyl, (C₁-C₄)alkylaminocarbonyl, di(C₁-C₄)alkylaminocarbonyl, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate.

Preferably, a heterocycloalkyl ring is unsubstituted or substituted with 1, 2, or 3 unsubstituted substituents selected from C₁-C₄ alkyl, C₁-C₄ haloalkyl, C₁-C₄ haloalkoxy, halogen, oxo, hydroxy, C₁-C₄ alkoxy, C₁-C₄ alkylthio, amino, carboxy, sulphate, phosphate and (C₁-C₄)alkylphosphate. More preferably, a heterocycloalkyl ring is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

A cycloalkyl group is a carbocyclic ring. The term carbocyclic ring means a cycloaliphatic group containing 3-10 carbon atoms such as, for example, cyclopropyl, cyclobutyl, cyclopentyl or cyclohexyl. Typically a carbocyclic ring is a 5- or 6-membered carbocyclic ring. Substituents which may be present on a carbocyclic ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate. Typically a carbocylclic ring is an unsubstituted carbocylclic ring or a carbocylclic ring substituted with 1, 2, or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, C₁-C₄ alkylamino, di(C₁-C₄)alkylamino, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate.

Preferably, a carbocyclic ring is unsubstituted or substituted with 1, 2, or 3 unsubstituted substituents selected from C₁-C₄ alkyl, C₁-C₄ haloalkyl, C₁-C₄ haloalkoxy, halogen, oxo, hydroxy, C₁-C₄ alkoxy, C₁-C₄ alkylthio, amino, sulphate, phosphate and (C₁-C₄)alkylphosphate. More preferably, a carbocyclic ring is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

For the avoidance of doubt, a fused heterocycloquinone group is a benzoquinone group fused to an heteroaryl or heterocycloalkyl ring, as defined above. Typically, a fused heterocycloquinone is a benzoquinone group fused to a 5- to 6-membered heteroaryl group or to a 5- to 6-membered heterocycloalkyl ring. Preferably, a fused heterocycloquinone is a benzoquinone group fused to a 5- to 6-membered heteroaryl group, for example a pyrrolyl group. An example of a fused heterocycloquinone group is a indole-4,7-dione-3-yl group.

Typically, the naphthoquinone or fused heterocycloquinone group is unsubstituted or substituted by one or more, for example, 1, 2, 3 or 4 substituents. Preferably, the naphthoquinone or fused heterocycloquinone group is unsubstituted or substituted by 1, 2 or 3 substituents. Typically, the benzoquinone group is unsubstituted or substituted by 1, 2 or 3 substituents. Typical substituents which may be present on the benzoquinone, naphthoquinone or fused heterocycloquinone group include C₁-C₆ alkyl, C₁-C₄ haloalkyl, C₁-C haloalkoxy, halogen, hydroxy, C₁-C₄ alkoxy, C₁-C₄ alkylthio, amino, C₁-C₄ alkylamino, di(C₁-C₄)alkylamino, heterocycloalkyl, cycloalkyl, aryl or heteroaryl. Preferred substituents are C₁-C₄ alkyl, C₁-C₄ haloalkyl, C₁-C₆ haloalkoxy, hydroxy, C₁-C₄ alkoxy and C₁-C₄ alkylthio. More preferred substituents are C₁-C₂ alkyl, C₁-C₂ haloalkyl, C₁-C₂ haloalkoxy, C₁-C₂ alkoxy and C₁-C₂ alkylthio. Typically the substituents are themselves unsubstituted.

A cytotoxic nucleoside analogue contains a substituted purine, pyrimidine or azapyrimidine ring, containing at least one amino substituent, and which ring is attached via a ring nitrogen atom to an unsubstituted or substituted five-membered saturated heterocyclic ring containing one or two oxygen or sulphur atoms such that the compound is an unnatural analogue of a natural nucleoside such as adenosine, cytidine, 2′-deoxyadenosine, 2′-deoxycytidine guanosine or 2′deoxyguanosine. Cytotoxic nucleoside analogues represented by R3NH₂ are known or can be determined by standard methods known to those skilled in the art. Such methods include in vitro assays of cell growth using cancer cell lines. Examples of such methods include DNA synthesis assays such as thymidine incorporation assays, protein stain assays such as sulphorhodamine B assays, vital stain assays such as neutral red assays, dye reduction assays such as MTT assays and dye exclusion assays such as trypan blue assays. Appropriate cytotoxic nucleoside analogues represented by R3NH₂ inhibit cell growth by at least 50% in one or more in vitro assays. Preferably the cytotoxic nucleoside analogues represented by R3NH₂ will inhibit cell growth by at least 50% in one or more in vitro assays at a concentration below 1 mM. Thus one skilled in the art can determine the group R3 in formula (1).

Where one or more functional groups in compounds of formula (1) are sufficiently basic or acidic the formation of salts is possible. Suitable salts include pharmaceutically acceptable salts for example acid addition salts including hydrochlorides, hydrobromides, phosphates, sulphates, hydrogen sulphates, alkylsulphonates, arylsulphonates, acetates, benzoates, citrates, maleates, fumarates, succinates, lactates and tartrates, salts derived from inorganic bases including alkali metal salts such as sodium or potassium salts, alkaline earth metal salts such as magnesium or calcium salts, and salts derived from organic amines such as morpholine, piperidine or dimethylamine salts.

Those skilled in the art will recognise that compounds of formula (1) may exist as stereoisomers and/or geometrical isomers and accordingly the present invention includes all such isomers which have anticancer activity and mixtures thereof.

Typically, in the compound of formula (1), R1 is a substituted aryl or 5- to 10-membered heteroaryl group bearing at least one nitro or azido group, or is a benzoquinone, naphthoquinone or fused heterocycloquinone. Typically, when R1 is a substituted aryl or 5- to 10-membered heteroaryl group bearing at least one nitro or azido group, it carries one substituent selected from a nitro or azido group and 0, 1 or 2 further unsubstituted substituents chosen from halogen, C₁-C₆ alkyl, hydroxy, amino, C₁-C₄ alkyamino, C₁-C₄ dialkyamino, C₁-C₄ haloalkyl, C₁-C₄ alkoxy and C₁-C₄ haloalkoxy substituents. Preferably, said further substituents are chosen from halogen, unsubstituted C₁-C₄ alkyl, hydroxy, and C₁-C₄ dialkyamino substituents. More preferably, said substituents are unsubstituted C₁-C₂ alkyl substituents. Typically, when R1 is a substituted aryl or 5- to 10-membered heteroaryl group bearing at least one nitro or azido group, it is a phenyl or a 5- to 6-membered heteroaryl group carrying one substituent selected from a nitro or azido group, and 0, 1 or 2 said further substituents More preferably, when R1 is a substituted aryl or 5- to 10-membered heteroaryl group bearing at least one nitro or azido substituent, said group carries only one substituent which substituent is chosen from a nitro or azido group. Preferably, said substituent is a nitro group.

More typically, when R1 is a substituted aryl or 5- to 10-membered heteroaryl group bearing at least one nitro or azido group, R1 is phenyl or a 5- or 6-membered heteroaryl group, for example a furanyl, imidazolyl or thienyl group, substituted by only one substituent which substituent is a nitro substituent. Particularly useful values of the moiety R1 include nitroimidazole groups, for example 2-nitroimidazol-5-yl and nitrothiophene groups, for example 5-nitrothien-2-yl. Further particularly useful examples of the moiety R1 include nitrofuranyl groups, for example 5-nitrofuran-2-yl.

Typically, when R1 is a benzoquinone, naphthoquinone or fused heterocycloquinone it is a 1,4-benzoquinone, a 1,4-naphthoquinone or an indole-4,7-dione. More typically when R1 is a benzoquinone, naphthoquinone or fused heterocycloquinone it is a 1,4-benzoquinon-2-yl, a 1,4-naphthoquinon-2-yl or an indole-4,7-dione-3-yl group. When R1 is a benzoquinone, naphthoquinone or fused heterocycloquinone such groups may be unsubstituted or have 1, 2, 3 or 4 substituents. Such substituents may be independently selected from alkyl, alkoxy, thioalkoxy, amino, alkylamino, dialkylamino, heterocycloalkyl, cycloalkyl, aryl or heteroaryl. Preferably the group R1 in formula (1) will have a one electron reduction potential at pH7 of between −200 to −550 mV, more preferably −250 to −500 mV. One electron reduction potentials, E(1), can be obtained from literature sources or measured by a number of methods known in the art. For example E(1) can be measured by pulse radiolysis by measuring the equilibrium constant for the electron transfer between the radical anions of the compound under study and an appropriate reference standard for example a viologen or quinone compound (Meisel, J Phys Chem 1975, 79, 1503-9).

Typically when R3 is a group of formula (2) A is N, CF or CH. More typically A is N or CH.

Typically when R3 is a group of formula (2) X is O or S. More typically X is O.

Typically when R3 is a group of formula (2) Y is CH₂, CHOH, CHOC(O)alkyl, CHF, CF₂, CHCN, C═CH₂, or C═CHF.

Typically when R3 is a group of formula (2) Z is CHOH, CR9′OH, CHOP(O)(OH)₂, CHOC(O)alkyl or O. More typically Z is CHOH, CHOP(O)(OH)₂, or CHOC(O)alkyl. Preferably Z is CHOH or CHOP(O)(OH)₂.

Typically when R3 is a group of formula (2) R4 is OH, OP(O)(OH)₂, OC(O)alkyl or H. More typically R4 is OH, OP(O)(OH)₂ or OC(O)alkyl.

Typically when R3 is a group of formula (3) X is O or S. More typically X is O.

Typically when R3 is a group of formula (3) Y is CH₂, CHOH, CHOC(O)alkyl, CHF, CF₂, CHCN, C═CH₂, or C═CHF. More typically Y is CH₂, CHOH, CHF or CF₂.

Typically when R3 is a group of formula (3) Z is CHOH, CR9′OH, CHOP(O)(OH)₂, CHOC(O)alkyl or O. More typically Z is CHOH, CHOP(O)(OH)₂ or CHOC(O)alkyl. Preferably Z is CHOH or CHOP(O)(OH)₂.

Typically when R3 is a group of formula (3) R5 is OH, OP(O)(OH)₂ or OC(O)alkyl. Preferably R5 is OH.

Typically when R3 is a group of formula (3) R6 and R7 are each independently H, Cl or F.

Typically when R3 is a group of formula (4) X is O or S. More typically X is O.

Typically when R3 is a group of formula (4) Y is CH₂, CHOH, CHOC(O)alkyl, CHF, CF₂, CHCN, C═CH₂, or C═CHF. More typically Y is CH₂, CHOH, CHF or CF₂. Typically when R3 is a group of formula (4) Z is CHOH, CR9′OH, CHOP(O)(OH)₂, CHOC(O)alkyl or O. More typically Z is CHOH, CHOP(O)(OH)₂ or CHOC(O)alkyl. Preferably Z is CHOH or CHOP(O)(OH)₂.

Typically when R3 is a group of formula (3) R5 is OH, OP(O)(OH)₂ or OC(O)alkyl. Preferably R5 is OH.

Typically when R3 is a group of formula (4) R8 is alkyl. Preferably. R8 is methyl.

One useful group of compounds of Formula (1) are those in which R3 is selected such that R3NH₂ represents gemcitabine, cytarabine (1-β-D-arabinofuranosylcytosine), fludarabine phosphate (2-fluoro-9-(5-O-phosphono-β-D-arabinofuranosyl)-9H-purin-6-amine), fludarabine (2-fluoro-9-(-β-D-arabinofuranosyl)-9H-purin-6-amine), cladribine (2-chloro-2′-deoxy-β-D-adenosine), troxacitabine (2′deoxy-3′oxacytidine), 5-azacytidine, decitabine (5-aza-2′-deoxycytidine), tezacitabine (E-2′deoxy-2-fluoromethylene)cytidine), DMDC (1-(2-deoxy-2-methylene-β-D-erythro-pentofuranosyl)cytosine), clofarabine (2-chloro-2′-fluoro-deoxy-9-β-D-arabinofuranosyladenine), fazarabine (1-β-D-arabinofuranosyl-5-azacytosine), vidarabine (9-β-D-arabinosyladenine), CNDAC (1-(2-C-cyano-2-deoxy-β-D-arabino-pentofuranosyl)-cytosine), OSI-7836 (4′-thio-aracytidine), 4-thio-FAC (1-(2-deoxy-2-fluoro-4-thio-β-D-arabinofuranosyl)cytosine), TAS-1061 ((3-C-ethynyl-β-D-ribo-pentofuranosyl)cytosine), ara-G (9-β-D-arabinofuranosyl guanine), nelarabine (2-amino-9-β-D-arabinofuranosyl-6-methoxy-9H-purine), 5′-deoxy-5-fluorocytidine, 2′,3′-di-O-acetyl-5′-deoxy-5-fluorocytidine and 2′,3′,5′-tri-O-acetyl-cytarabine.

In a preferred embodiment, R1 is either:

(a) a phenyl or 5- to 10-membered heteroaryl group bearing at least one nitro or azido group and 0, 1 or 2 further unsubstituted substituents selected from halogen, C₁-C₆ alkyl, hydroxy, amino, C₁-C₄ alkylamino, di(C₁-C₄)alkylamino, carboxy, (C₁-C₄)alkylthio, C₁-C₄ alkoxy, C₁-C₄ haloalkyl, and C₁-C₄ haloalkoxy; or

(b) a benzoquinone group which is unsubstituted or substituted by 1, 2 or 3 unsubstituted substituents or a naphthoquinone or fused heterocycloquinone group which is unsubstituted or substituted by 1, 2, 3 or 4 unsubstituted substituents, said unsubstituted substituents being selected from C₁-C₆ alkyl, C₁-C₄ haloalkyl, C₁-C₆ haloalkoxy, hydroxy, C₁-C₄ alkoxy and C₁-C₄ alkylthio.

Preferably, when R1 is as defined above in (a), said further unsubstituted substituents are selected from C₁-C₂ alkyl, C₁-C₂ haloalkyl, C₁-C₂ alkoxy and C₁-C₂ haloalkoxy substituents. More preferably, said further unsubstituted substituents are selected from C₁-C₂ alkyl. Preferably, when R1 is as defined above in (a), it is a phenyl or 5- to 6-membered heteroaryl group carrying one substituent selected from a nitro or azido group and 0, 1 or 2 said further unsubstituted substituents. More preferably, when R1 is as defined above in (a), it is a substituted phenyl or 5- to 6-membered heteroaryl group bearing only one nitro or azido substituent and 0 or 1 said further unsubstituted substituents. More preferably, when R1 is as defined above in (a), it is a phenyl or 5- to 6-membered heteroaryl group bearing only one nitro substituent and 0 or 1 said further unsubstituted substituents. In one embodiment, R1 is a phenyl or 5-membered heteroaryl group bearing at least one nitro or azido substituent.

Preferably, when R1 is as defined above in (b), said substituents are selected from unsubstituted C₁-C₂ alkyl, C₁-C₂ haloalkyl, C₁-C₂ haloalkoxy, C₁-C₂ alkoxy and C₁-C₂ alkylthio groups. Preferably, when R1 is as defined above in (b), it is a benzoquinone, naphthoquinone or a fused heterocycloquinone group wherein a benzoquinone group is fused to a 5- to 6-membered heteroaryl group, which is unsubstituted or substituted by 1, 2, or 3 said unsubstituted substituents.

In a preferred embodiment, R2 is typically an H or an unsubstituted C₁-C₆ alkyl, C₂-C₆ alkenyl, C₂-C₆ alkynyl, C₃-C₁₀ cycloalkyl, 5- to 6-membered heterocycloalkyl, phenyl or a 5- to 10-membered heteroaryl group. Preferably R2 is H or an unsubstituted C₁-C₄ alkyl group. More preferably, R2 is H or an unsubstituted C₁-C₂ alkyl group.

In a preferred embodiment, A is preferably CH or CF. In one embodiment, A is CH.

In a preferred embodiment, X is preferably O.

In a preferred embodiment, Y is typically CH₂, CHOH, CHO(CO)alkyl, CHF, CF₂, C═CH₂, or C═CHF. Preferably, Y is an unsubstituted CH₂, CHOH, CHO(CO)—C₁-C₆ alkyl, CHF or CF₂ group. More preferably Y is an unsubstituted CH₂, CHOH, CHO(CO)—C₁-C₄, CHF or CF₂ group. It is further preferred that Y is an unsubstituted CH₂, CHOH, CHO(CO)—C₁-C₂ alkyl or CF₂ group. In one embodiment, Y is preferably an unsubstituted CH₂, CHOH, CHO(CO)—C₁-C₆ alkyl or CHF group.

In a preferred embodiment, Z is typically an unsubstituted CHOH, CR9′OH, CHOP(O)(OH)₂, CHOC(O)—C₁-C₆ alkyl or O group. Preferably, Z is an unsubstituted CHOH or CHOC(O)—C₁-C₄ alkyl group. More preferably, Z is an unsubstituted CHOH or CHOC(O)—C₁-C₂ alkyl group.

In a preferred embodiment, R4 is typically H or an unsubstituted OH, OP(O)(OH)₂ or OC(O)—C₁-C₆ alkyl group. Preferably R4 is H or an unsubstituted OH or OC(O)—C₁-C₄ alkyl group. More preferably, R4 is H or an unsubstituted OH or OC(O)—C₁-C₂ alkyl group. In one embodiment, R4 is an unsubstituted OH, OP(O)(OH)₂ or OC(O)—C₁-C₆ alkyl group. In a further embodiment, when R3 is a group of formula (2), either: (a) A is CH; or (b) R4 is an unsubstituted OH, OP(O)(OH)₂ or OC(O)—C₁-C₆ alkyl group.

In a preferred embodiment, R5 is typically H or an unsubstituted OH, OP(O)(OH)₂ or OC(O)—C₁-C₆ alkyl group. Preferably R5 is H or an unsubstituted OH or OC(O)—C₁-C₄ group. More preferably, R5 is H or an unsubstituted OH or OC(O)—C, C₂ alkyl group. It is particularly preferred that R5 is OH.

In a preferred embodiment, R6 is typically H.

In a preferred embodiment, R7 is typically H or F.

In a preferred embodiment, R8 is typically H or an unsubstituted C₁-C₆ alkyl group. Preferably, R8 is H or an unsubstituted C₁-C₄ alkyl group. More preferably R8 is H or an unsubstituted C₁-C₂ alkyl group.

In a preferred embodiment, R9′ is typically an unsubstituted C₁-C₆ alkyl, C₂-C₆ alkenyl, or C₂-C₆ alkynyl group. Preferably R9′ is an unsubstituted C₂-C₄ alkynyl group. More preferably, R9′ is an unsubstituted ethynyl group.

In a preferred embodiment, R3 is typically a group of formula (2), (3) or (4) wherein A, X, Y, Z and R4 to R8 and R9′ are as defined above. Preferably, R3 is a group of formula (2) or (3).

In a more preferred embodiment, in the compound of formula (1),
R1 is either:
(a) a phenyl or 5- to 10-membered heteroaryl group bearing at least one nitro or azido group and 0, 1 or 2 further unsubstituted substituents selected from halogen, C₁-C₆ alkyl, hydroxy, amino, C₁-C₄ alkylamino, di(C₁-C₄)alkylamino, carboxy, (C₁-C₄)alkylthio, C₁-C₄ alkoxy, C₁-C₄ haloalkyl, and C₁-C₄ haloalkoxy; or
(b) a benzoquinone group which is unsubstituted or substituted by 1, 2 or 3 unsubstituted substituents or a fused heterocycloquinone or naphthoquinone group which group is unsubstituted or substituted by 1, 2, 3 or 4 unsubstituted substituents, which unsubstituted substituents are selected from C₁-C₆ alkyl, C₁-C₄ haloalkyl, C₁-C₆ haloalkoxy, hydroxy, C₁-C₄ alkoxy and C₁-C₄ alkylthio;
   R2 is H or an unsubstituted C₁-C₆ alkyl, C₂-C₆ alkenyl, C₂-C₆ alkynyl, C₃-C₁₀ cycloalkyl, 5- to 6-membered heterocycloalkyl, phenyl or a 5- to 10-membered heteroaryl group;
   A is CH or CF;
   X is O;
   Y is an unsubstituted CH₂, CHOH, CHO(CO)—C₁-C₆ alkyl, CHF or CF₂ group;
   Z is an unsubstituted CHOH, CR9′OH, CHOP(O)(OH)₂, CHOC(O)—C₁-C₆ alkyl or O group;
   R4 is H or an unsubstituted OH, OP(O)(OH)₂ or OC(O)—C₁-C₆ alkyl group;
   R5 is H or an unsubstituted OH, OP(O)(OH)₂ or OC(O)—C₁-C₆ alkyl group;
   R6 is H;
   R7 is H or F;
   R8 is H or an unsubstituted C₁-C₆ alkyl group;
   R9′ is an unsubstituted C₁-C₆ alkyl, C₂-C₆ alkenyl, or C₂-C₆ alkynyl group; and
   R3 is a group of formula (2), (3) or (4) wherein A, X, Y, Z and R4 to R8 and R9′ are as defined above.

In a further preferred embodiment, in the compound of formula (1),
either R1 is:
   (a) a phenyl or 5- to 6-membered heteroaryl group carrying only one nitro substituent and 0 or 1 further unsubstituted substituents selected from C₁-C₂ alkyl; or
   (b) a benzoquinone, naphthoquinone or a fused heterocycloquinone group wherein a benzoquinone group is fused to a 5- to 6-membered heteroaryl group, which is unsubstituted or substituted by 1, 2, or 3 unsubstituted substituents selected from C₁-C₂ alkyl, C₁-C₂ haloalkyl, C₁-C₂ haloalkoxy, C₁-C₂ alkoxy and C₁-C₂ alkylthio;
R2 is H or an unsubstituted C₁-C₂ alkyl group
A is CH or CF;
X is O;
Y is an unsubstituted CH₂, CHOH, CHO(CO)—C₁-C₂ alkyl or CF₂ group;
Z is an unsubstituted CHOH or CHOC(O)—C₁-C₂ alkyl group;
R4 is H or an unsubstituted OH or OC(O)—C₁-C₂ alkyl group;
R5 is OH;
R6 is H;
R7 is H or F; and
R3 is a group of formula (2) or (3) wherein A, X, Y, Z and R4 to R7 and R9′ are as defined above.

Most preferably, the compound of formula (1) is selected from:
N⁴-(1-(5-nitrothien-2-yl)ethyl)oxycarbonyl-1-β-D-arabinofuranosylcytosine;
N⁴-(5-nitrothien-2-yl)methoxycarbonyl-1-β-D-arabinofaranosylcytosine;
N⁴-(1-(5-nitrothien-2-yl)ethyl)oxycarbonyl-2′,2′-difluoro-2′-deoxycytidine;
Tri-O-acetyl-N⁴-(1-(5-nitrothien-2-yl)ethyl)oxycarbonyl-1-β-D-arabinofuranosylcytosine;
N⁴-(2-nitro-1-methylimidazol-5-yl)methoxycarbonyl-2′,2′-difluoro-2′-deoxycytidine;
N⁴-(5-nitrothien-2-yl)methoxycarbonyl-2′,2′-difluoro-2′-deoxycytidine;
N⁴-(5-nitro-1-methylimidazol-2-yl)methoxycarbonyl-1-β-D-arabinofuranosylcytosine;
N⁶-(5-nitrothien-2-yl)methoxycarbonyl-9-β-D-arabinofaranosyladenine;
5′-Deoxy-2′,3′-di-O-acetyl-5-fluoro-N⁴-((5-nitrothien-2-yl)methoxycarbonyl)cytidine;
2-fluoro-N-(5-nitrothien-2-yl)methoxycarbonyl-9-β-D-arabinofuranosyladenine;
N⁴-(1-(4-nitrophenyl)ethoxycarbonyl)-1-β-D-arabinofuranosylcytosine;
N⁴-(5-nitrofuran-2-yl)methoxycarbonyl-1-β-D-arabinofuranosylcytosine;
N⁴-(5-methoxy-1,2-dimethyl-4,7-dioxoindol-3-yl)methoxycarbonyl-1-β-D-arabinofuranosylcytosine; and
5′-Deoxy-5-fluoro-N⁴-((5-nitrothien-2-yl)methoxycarbonyl)cytidine.

It is a further object of this invention to provide methods for the preparation of compounds of formula (1).

Compounds of formula (1) may be prepared by a number of processes as generally described below and more specifically in the Examples hereinafter. In the following process description, the symbols R1, R2 and R3 when used in the formulae depicted are to be understood to represent those groups described above in relation to formula (1) unless otherwise indicated. In the schemes described below it may be necessary to employ protecting groups that are then removed during the final stages of the synthesis. The appropriate use of such protecting groups and processes for their removal will be readily apparent to those skilled in the art.

Compounds of formula (1) may be prepared for example by reaction of a compound of formula (5) wherein R9 is a leaving group, for example a halogen such as fluoro, chloro or bromo or for example a nitrophenol such as 4-nitrophenol or for example imidazole, with a protected or unprotected nucleoside analogue for example a compound of formula R3NH₂, in a solvent such as a chlorinated solvent for example dichloromethane, optionally in the presence of a base such as an amine base for example triethylamine, pyridine, at a temperature of from about −20° C. to the reflux temperature of the solvent, followed if necessary by deprotection. Suitable protecting groups for hydroxy groups in the nucleoside analogue include silicon protecting groups such as trimethylsilyl or t-butyldimethylsilyl. Suitable protecting groups for hydroxy groups in the nucleoside analogue also include alkyl carbonyl groups such as acetyl, and alkyloxycarbonyl groups such as t-butyloxycarbonyl. Compounds of formula R3NH₂ are either known or can be prepared by standard methods apparent to one skilled in the art.

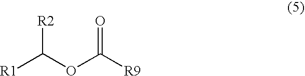

Compounds of formula (5) can be prepared from alcohols of formula (6) by treatment with compounds of formula (7) in which R9 and R10, which may be the same or different are leaving groups. Examples of typical leaving groups are a halogen such as fluoro, chloro or bromo or for example a nitrophenol such as 4-nitrophenol or for example imidazole. Compounds of formula (7) are either known or can be prepared by standard methods apparent to one skilled in the art.

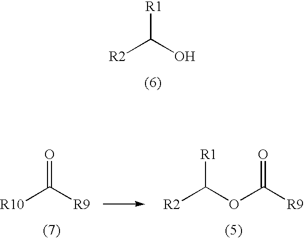

Alcohols of formula (6) are either known or can be prepared by standard methods apparent to one skilled in the art. Such methods include treatment of an aldehyde or ketone of formula (8) with a reducing agent, for example a borohydride reducing agent such as sodium borohydride in a solvent such as an alcoholic solvent for example methanol at a temperature between about −20° C. to room temperature, preferably around 0° C. Such methods also include the treatment of an aldehyde of formula (9) with an organometallic compound of formula (10) in which M represents a metal, metal halide or dialkylmetal, for example, Li, ZiBr, MgBr or MgI or dialkylaluminium in a solvent such as an ether solvent, for example tetrahydrofuran or diethyl ether or in an aromatic solvent for example benzene or toluene at a temperature of between about −78° C. to about the reflux temperature of the solvent, preferably from about 0° C. to room temperature. Where Ar is a substituted aryl or heteroaryl group bearing at least one nitro group such methods also include the aromatic electrophilic nitration of the appropriate aryl substrate with an appropriate nitrating agent at a temperature of between about −78° C. and room temperature. Appropriate nitrating agents are, for example, nitric acid in a solvent such as an acid anhydride for example acetic anhydride or in a solvent such as an acid for example sulphuric acid or acetic acid; nitronium tetrafluoroborate in a solvent such as an ether solvent, for example tetrahydrofuran or diethyl ether or in a solvent such as acetonitrile or glacial acetic acid or in a solvent such as a chlorinated solvent for example dichloromethane or dinitrogen tetroxide in a solvent such as an ether solvent, for example tetrahydrofuran or diethyl ether or in a solvent such as acetonitrile or glacial acetic acid or in a solvent such as a chlorinated solvent for example dichloromethane or in an aromatic solvent for example benzene or toluene.

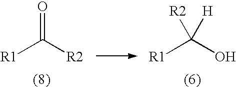

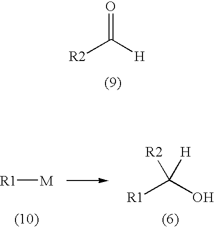

Compounds of formula (1) can also be prepared in a one-pot procedure by treatment of a suitably protected compound of formula R3NH₂ with an alcohol of formula (6) and phosgene in a solvent such as a chlorinated solvent for example dichloromethane in the presence of a base such as an amine base for example pyridine at a temperature of between about −20° C. and the boiling point of the solvent, preferably between −5° and room temperature.

Compounds of formula (1) can also be prepared from other compounds of formula (1) by functional group transformation. Such transformations include standard hydrolysis, oxidation, reduction and substitution reactions. For example a compound of formula (1) containing one or more acetyl groups can be converted into the corresponding compound of formula (1) containing one or more hydroxyl groups by ester hydrolysis. Such hydrolysis can be affected for example by enzymatic hydrolysis with an esterase such as porcine liver esterase.

Preparation of a compound of formula (1) as a single enantiomer or, where appropriate, diastereomer, may be effected by synthesis from an enantiomerically pure starting material or intermediate or by resolution of the final product in a conventional manner.

The compounds of the invention may be administered as a sole therapy or in combination with other treatments. For the treatment of solid tumours compounds of the invention may be administered in combination with radiotherapy or in combination with other anti-tumour substances for example those selected from mitotic inhibitors, for example vinblastine, vincristine, vinorelbine, paclitaxel and docetaxel; alkylating agents, for example cisplatin, carboplatin, oxaliplatin, nitrogen mustard, melphalan, chlorambucil, busulphan and cyclophosphamide; antimetabolites, for example 5-fluorouracil, cytosine arabinoside, gemcitabine, capecitabine, methotrexate and hydroxyurea; intercalating agents for example adriamycin and bleomycin; enzymes, for example aspariginase; topoisomerase inhibitors for example etoposide, teniposide, topotecan and irinotecan; thymidylate synthase inhibitors for example raltitrexed; biological response modifiers for example interferon; antibodies for example edrecolomab, cetuximab, bevacizumab and trastuzumab; receptor tyrosine kinase inhibitors for example gefitinib, imatinib and erlotinib; and anti-hormones for example tamoxifen, anastrazole, exemestane and letrozole. Such combination treatment may involve simultaneous or sequential application of the individual components of the treatment.

For the prophylaxis and treatment of disease the compounds according to the invention may be administered as pharmaceutical compositions selected with regard to the intended route of administration and standard pharmaceutical practice. Such pharmaceutical compositions may take a form suitable for oral, buccal, nasal, topical, rectal or parenteral administration and may be prepared in a conventional manner using conventional excipients. For example for oral administration the pharmaceutical compositions may take the form of tablets or capsules. For nasal administration or administration by inhalation the compounds may be conveniently delivered as a powder or in solution. Topical administration may be as an ointment or cream and rectal administration may be as a suppository. For parenteral injection (including intravenous, subcutaneous, intramuscular, intravascular or infusion) the composition may take the form of, for example, a sterile solution, suspension or emulsion.

The dose of a compound of the invention required for the prophylaxis or treatment of a particular condition will vary depending on the compound chosen, the route of administration, the form and severity of the condition and whether the compound is to be administered alone or in combination with another drug. Thus the precise dose will be determined by the administering physician but in general daily dosages may be in the range 0.001 to 100 mg/kg preferably 0.1 to 10 mg/kg. Typically, daily dosage levels are from 0.05 mg to 2 g, for example from 5 mg to 1 g.

The compounds of the present invention are therapeutically useful in treating, preventing, ameliorating or reducing incidence of a proliferative disorder. Typically, the proliferative disorder is a hypoxic disorder. A hypoxic disorder is typically a disorder in which diseased cells are present in a hypoxic environment. Examples of the disorders that can be treated, prevented, ameliorated or disorders whose incidence can be reduced, include cancer, rheumatoid arthritis, psoriatic lesions, diabetic retinopathy or wet age-related macular degeneration.

Typically, the disorder is cancer. Preferably the cancer is a hypoxic cancer. A hypoxic cancer is, of course, a cancer wherein cancerous cells are in a hypoxic environment. Most preferably, the cancer is a solid tumour or leukaemia. Typically the leukaemia is leukaemia involving the spleen or bone marrow.

According to a further aspect of the invention there is provided a compound of formula (1), or a pharmaceutically acceptable salt or solvate thereof, for use in a method of treatment of the human or animal body by therapy. In particular, the present invention provides a method of ameliorating or reducing the incidence of a said proliferative disorder in a patient, which method comprises administering to said patient an effective amount of a compound of formula (1), or a pharmaceutically acceptable salt or solvate thereof.

A further feature of the present invention is a compound of formula (1), or a pharmaceutically acceptable salt or solvate thereof, for use as a medicament. In particular, the present invention provides a compound of formula (1), or a pharmaceutically acceptable salt thereof, for the treatment of the human or animal body.

According to a further aspect of the invention there is provided the use of a compound of formula (1), or a pharmaceutically acceptable salt or solvate thereof, in the manufacture of a medicament for use in the therapy of a warm-blooded animal, for example a human, suffering from a proliferative disease for example cancer. In particular, the present invention provides the use of a compound of formula (1), or a pharmaceutically acceptable salt thereof, in the manufacture of a medicament for use in the treatment of the human or animal body, for the prevention or treatment of a said proliferative disorder.

A number of enzymes are capable of reducing aryl and heteroaryl nitro groups. Strategies that increase the activity of such enzymes within solid tumours can therefore increase further the activity of prodrugs dependent on nitro reduction. Similarly a number of enzymes are capable of reducing quinones and indoloquinones and therefore similar strategies are possible to increase the effectiveness of drugs requiring activation by quinone reduction. Such strategies include linking such enzymes to a tumour-targeting antibody, administering such enzyme antibody conjugates to a host with a solid tumour then, after the conjugate has localised to the tumour, administering the prodrug. This approach is known as Antibody Directed Enzyme Prodrug Therapy (ADEPT). Alternatively the gene encoding for the enzyme might be delivered selectively and/or expressed selectively, in the tumour before administration of the prodrug. This approach is known as Gene Directed Enzyme Prodrug Therapy (GDEPT). When the gene is delivered by a viral vector the approach is sometimes known as Virus Directed Enzyme Prodrug Therapy (VDEPT).

Anlezark has disclosed nitroreductases and their use in an ADEPT strategy. Prodrugs for use in this strategy were also disclosed (U.S. Pat. No. 5,633,158 and U.S. Pat. No. 5,977,065). In WO 00 047725 Anlezark provides further disclosures of nitroreductase enzymes and their use in GDEPT strategies. Denny (WQ 00 064864) has disclosed nitroaryl and nitroheteroaryl prodrugs for use in a GDEPT strategy. The use of quinone-reducing enzymes in ADEPT, GDEPT and MDEPT (Macromolecule Directed Enzyme Prodrug Therapy) is discussed in Skelly et al. Mini Rev Med. Chem. 2001, 1, 293-306.

Thus it is a further object of this invention to provide the use of compounds of formula (1) in combination with a reductase, an antibody-reductase conjugate, a macromolecule-reductase conjugate or DNA encoding a reductase gene, in a method of treatment for the human body. Thus, the present invention provides a method of ameliorating or reducing the incidence of a said proliferative disorder in a patient, which method comprises administering to said patient an effective amount of
(a) a compound of formula (1), or a pharmaceutically acceptable salt thereof; and
(b) a reductase, an anti-body reductase conjugate, a macromolecule-reductase conjugate or DNA encoding a reductase gene.

Further, the present invention provides a product containing
(a) a compound of formula (1), or a pharmaceutically acceptable salt thereof; and
(b) a reductase, an anti-body reductase conjugate, a macromolecule-reductase conjugate or DNA encoding a reductase gene for simultaneous, separate or sequential use in the treatment of a proliferative condition.

The ability of compounds of the invention to release cytotoxic or cytostatic agents selectively under hypoxic conditions can be assessed by using, for example, one or more of the procedures set out below:

### Radiolysis

In the hypoxic environments of solid tumours, prodrugs can be reduced by one-electron processes that are inhibited in the normoxic environments of normal tissues. Radiolysis demonstrates the ability of bioreductively-activated prodrugs to release the active drug after one-electron reduction. Compounds were dissolved in an isopropanol/water mixture (50:50) at a concentration of 50 μM or below. Solutions, in gas-tight syringes, were saturated with nitrous oxide before irradiation in a ⁶⁰Co source at a dose rate of 3.9 Gy min⁻¹ (as determined by Fricke dosimetry: H. Fricke and E. J. Hart, “Chemical Dosimetry” in Radiation Dosimetry Vol. 2 (F. H. Attrix and W. C. Roesch. Eds.), pp 167-239. Academic Press New York, 1966.). Solutions were analyzed for released drug by HPLC. In this test example compounds of the invention produced cytotoxic nucleosides (or their ester prodrugs) efficiently with radiation chemical yields (G-value) as shown in Table 1.

TABLE 1
Radiation chemical yields of cytotoxic nucleosides released by gamma
radiolysis

| Compound | | |
|---|---|---|
| of Example | Drug released | G (μmol · J⁻¹) |
| 1 | cytarabine | 0.50 |
| 2 | cytarabine | 0.43 |
| 3 | gemcitabine | 0.69 |
| 4 | triacetylcytarabine | 0.67 |
| 5 | gemcitabine | 0.29 |
| 6 | gemcitabine | 0.71 |
| 8 | vidarabine | 1.00 |
| 9 | 5′-Deoxy-2′,3′-di-O- | 0.60* |
| | acetyl-5-fluoro-cytidine | |
| 11 | cytarabine | 0.63 |
| 12 | cytarabine | 0.74 |
| 13 | cytarabine | 3.94 |

*rate of prodrug loss

### Pulse Radiolysis

The rate of fragmentation of intermediate radical anions produced by one-electron reduction is an important determinant of bioreductive prodrug action and can be measured by pulse radiolysis. The magnitude of the rate constant of radical fragmentation indicates the ability of the prodrugs to deliver the drug under hypoxia. The radicals were generated by reduction of the parent prodrugs (40 μM) by the 2-propanol radical generated radiolytically in an N₂O-saturated 50% aqueous 2-propanol buffered to pH 7.4-9.0 with potassium phosphate (4 mM). Experiments were performed with a 6 Me V linear accelerator to generate an electron pulse of around 500 ns. The absorbed radiation dose per electron pulse (typically 5-35 Gy was determined by the thiocyanate dosimeter. Changes in absorbance were measured with a tungstaen lamp and photodiode detector preceded by a single-pass monochromator. Compounds of the invention had good rate constants (k Table 2) in contrast to the prior art compound N⁴-(4-nitrophenyl)methoxycarbonyl-1-β-D-arabinofuranosylcytosine (Fadl et al Pharmazie. 1995, 50, 382-7), the radical anion of which decayed very slowly in this test and the rate constant of fragmentation was too small to measure.

TABLE 2
First-order rate constants for the decay of radical anions produced
by pulse radiolysis of compounds of the invention.

| Compound of Example | k (s⁻¹) |
|---|---|
| 1 | 49 |
| 2 | 12 |
| 3 | 102 |
| 4 | 245 |
| 5 | 18 |
| 6 | 20 |
| 8 | 148 |
| 9 | 22 |
| 10 | 144 |
| 11 | 0.4 |
| 12 | 5.5 |
| 13 | 68 |

### Metabolism in Tumour Homogenates

Useful bioreductive prodrugs can be shown to release the active drug selectively under conditions of low oxygen in the presence of tumour homogenate in this assay. Freshly-excised CaNT or FaDu tumours (approximately 0.5 to 1 g) were homogenised in 15 ml of ice-cold 50 mmol dm-3 potassium phosphate buffer at pH 7.4. The homogenates were centrifuged at 1000 RPM for 10 min and the supernatants stored on ice. The metabolism of 5 μmol dm⁻³ prodrug in air and N₂ was performed with 0.5 ml tumour homogenate (a 3 mg of protein by Bradford assay) with 100 μmol dm³ NADPH in 50 mmol dm⁻³ potassium phosphate buffer at pH 7.4 incubated at 37° C. Samples (60 pt) were taken at regular intervals and added to an equivalent volume of acetonitrile, then mixed and centrifuged at 14, 300 RPM for 2 min prior to product analysis by HPLC. In this assay, using FaDu tumours, the compound of Example (1) released cytarabine at a rate of 0.29 nmol min⁻¹ mg protein⁻¹ under nitrogen but at only 0.02 mmol min⁻¹ mg protein⁻¹ under air.

### Cellular Cytotoxicity

In a preferred embodiment of the invention the compounds of Formula (1) will be less potent as cytotoxic agents than the corresponding cytotoxic nucleosides of formula R3NH₂ which are released under hypoxic conditions. The cytotoxic or cytostatic properties of compounds of Formula (1) and compounds of Formula R3NH₂ can be assessed for example, by use, for example, of this assay. The Celltiter 96® Aqᵤₑₒᵤₛ One Solution Cell Proliferation Assay kit (Promega Corporation, USA) which is a calorimetric method for determining the number of viable cells in proliferation or cytotoxicity assays was used. In this assay the MTS tetrazolium compound (Owen's Reagent) is bioreduced by viable cells into a coloured formazan product which is soluble in tissue culture medium and can be measured by recording absorbance at 490 um with a 96 well plate reader. A549 cells were seeded in Eagles Minimum Essential Medium supplemented with 10% foetal calf serum and non-essential amino acids at 10³ cell per well on a 96 well plate and allowed to attach for 24 h. Compounds were dissolved in DMSO and diluted with cell culture medium before addition. The cells were exposed to test compound for 48 h. The MTS reagent was added to each well, left for 4 h, then the absorbance measured at 490 nm with a 96 well plate reader.

### Metabolism in Liver Homogenates

Release of parent drug from bioreductive prodrugs under hypoxia can be demonstrated using liver homogenates as a source of the reductase enzymes also present in solid tumours. Metabolic stability of the compounds and unfavorable release of the drug by oxic liver can also be assessed by using this assay. Freshly-excised mouse liver (approximately 1 g) was homogenised in 15 ml of ice-cold 50 mmol dm⁻³ potassium phosphate buffer at pH 7.4. The homogenates were centrifuged at 1000 RPM for 10 min and the supernatants stored on ice. The metabolism of 5 μmol dm⁻³ prodrug in air was performed with 0.5 ml liver homogenate (˜2 mg of protein by Bradford assay) with 100 μmol dm⁻³ NADPH in 50 mmol dm⁻³ potassium phosphate buffer at pH 7.4 incubated at 37° C. Samples (60 μl) were taken at regular intervals and added to an equivalent volume of acetonitrile, then mixed and centrifuged at 14, 300 RPM for 2 min prior to product analysis by HPLC. Example compounds of the invention efficiently released cytotoxic nucleoside analogues under nitrogen (anoxic) but the release under air (oxic) was much slower.

TABLE 3
Oxygen-inhibited release of drugs from example compounds
of the invention catalysed by liver homogenate

| | | |
|---|---|---|
| | | Rate of |
| | | drug release |
| | | (nmol/min/ |
| Compound | Drug | mg protein) |

| of Example | released | Anoxic | Oxic |
|---|---|---|---|
| 1 | cytarabine | 1.68 | 0.15 |
| 2 | cytarabine | 1.56 | 0.06 |
| 3 | gemcitabine | 1.24 | 0.66 |
| 4 | cytarabine | 4.37 | 0.38 |
| 5 | gemcitabine | 0.67 | 0.002 |
| 8 | vidarabine | 3.22 | 0.056 |
| 12 | cytarabine | 1.47 | 0.000 |

In contrast to the efficient release of drugs from compounds of the invention, the release of cytarabine from the prior art compound N⁴-(4-nitrophenyl)methoxycarbonyl-1-β-D-arabinofuranosylcytosine (Fadl et al Pharmazie. 1995, 50, 382-7) was only 0.12 nmol/min/mg protein under nitrogen.
Drug Release by Cytochrome p450 Reductase

Cytochrome p450 reductase is widely expressed in human tumours as well as in a range of normal tissues and is one of a number of enzymes that can catalyse bioreduction. This assay shows the ability of prodrugs to fragment into active drugs catalysed by cytochrome p450 selectively under conditions of low oxygen. Compounds were dissolved in DMSO to a concentration of 625 μM and 20 μL added to a mixture of 50 mmol dm⁻³ potassium phosphate buffer at pH 7.4 (2.4 mL), NADPH (20 μL of a 10 mM solution) and 60 μL of Supersomal™ human P450 reductase (Gentest; Catalogue number P244) or 25 μL of bactosomal human P450 reductase (Cypex; Catalogue number Cyp004) and incubated at 37° C. For experiments under nitrogen the mixture was degassed with nitrogen for 20 minutes prior to compound addition and overgassed with nitrogen during the incubation. Samples (100 μl) were taken at regular intervals and added to an equivalent volume of acetonitrile, then mixed and centrifuged at 14, 300 RPM for 2 min prior to product analysis by HPLC.

The invention is illustrated by the following non-limiting Examples in which, unless otherwise stated:

DMF means dimethylformamide
DMSO means dimethylsulphoxide
THF means tetrahydrofuran
EtOAc means ethyl acetate
DCM means dichloromethane
TLC means thin-layer chromatography
TFA means trifluoroacetic acid

### EXAMPLE 1

### N⁴-(1-(5-nitrothien-2-yl)ethyl)oxycarbonyl-1-β-D-arabinofuranosylcytosine

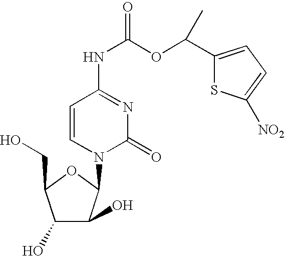

Tri-O-acetyl-N⁴-(1-(5-nitrothien-2-yl)ethyl)oxycarbonyl-1-β-D-arabinofaranosylcytosine (360 mg, 0.633 mmol) was dissolved in DMSO (5 mL) then phosphate buffer (20 mL, pH7) was added and a precipitate formed in situ. The mixture was warmed to 34° C., followed by addition of pig liver esterase (50 mg). Further 50 mg portions of esterase were added at 24 h and 48 h. After 72 h, the reaction mixture was partitioned (ethyl acetate and brine), the aqueous phase was extracted (ethyl acetate), the organic phases were combined, washed (water then brine) then dried (Na₂SO₄) and adsorbed onto flash silica. Flash chromatography, eluting with ethyl acetate then 5% and 10% methanol/ethyl acetate, afforded a colourless oil (20 mg, 7%); TLC R_{f}=0.5, 10% methanol/ethyl acetate. NMR (500 Mhz, DMSO-d6, δ) 10.95 (1H, s, NH), 8.09 (2H, s, HarH), 7.32 (1H, s, HarH), 6.99 (1H, s, NCH═CH), 6.12 (1H, s, NCHO), 6.07 (1H, d, J=6.5, OCHCH₃), 5.56 (2H, s, 2×OH), 5.10 (1H, s, OH), 4.06 (1H, bs, CHOH), 3.93 (1H, bs, OCHCH₂OH), 3.84 (1H, bs, CHOH), 3.66 (2H, m, CH₂OH), 1.65 (3H, d, J=6.5, OCHCH₃) ppm.

### EXAMPLE 2

### N⁴-(5-nitrothien-2-yl)methoxycarbonyl-1-β-D-arabinofuranosylcytosine

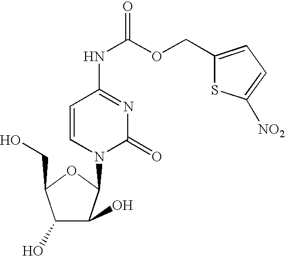

Chlorotrimethylsilane (76 μL, 0.60 mmol) was added to Ara-C (48 mg, 0.20 nmol), pyridine (97 μL, 1.20 mmol) and DCM (0.3 mL) at 0° C. After 2 h, 5-nitrothien-2-ylmethanol (48 mg, 0.30 mmol) in DCM (0.2 mL) was added, followed by dropwise addition of phosgene solution (0.2 mL, 0.24 mmol, 2M in toluene). The reaction was stirred for a further 18 h. The crude mixture was partitioned (ethyl acetate and brine), the aqueous phase was extracted (ethyl acetate); the organic phases were combined, washed (water then brine) then adsorbed on to flash silica in vacuo. Flash chromatography, eluting with DCM then 2%, 5%, and 10% methanol/ethyl acetate, afforded the title compound as a white wax (7 mg, 8%); TLC R_{f}=0.3, 10% methanol/ethyl acetate. ¹H NMR (500 Mhz, DMSO-d₆, δ) 10.98 (1H, s, NH), 8.10 (2H, s, HarH), 7.35 (1H, s, HarH), 7.07 (1H, s, NCH═CH), 6.08 (1H, s, NCHO), 5.51 (2H, s, 2×OH), 5.43 (2H, s, HarCH₂O), 5.05 (1H, s, OH), 4.10 (1H, bs, CHOH), 3.95 (1H, bs, OCHCH₂OH), 3.85 (1H, bs, CHOH), 3.61 (2H, m, CH₂OH) ppm.

### EXAMPLE 3

### N⁴-(1-(5-nitrothien-2-yl)ethyl)oxycarbonyl-2′,2′-difluoro-2′-deoxycytidine

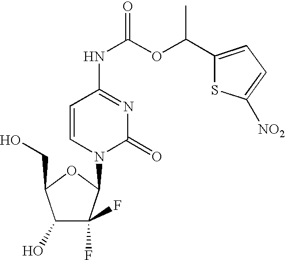

3′,5′-Di-O-t-butoxycarbonyl-N⁴-(1-(5-nitrothien-2-yl)ethyl)oxycarbonyl-2′,2′-difluoro-2′-deoxycytidine (133 mg, 0.20 mmol) was dissolved in DCM (14 mL) then cooled to 0° C. A solution of TFA (2 mL) and DCM (1 mL) was added dropwise to the cooled reaction mixture and stirring continued for 3 h. A further aliquot of TFA (1 mL) was added slowly, and reaction was complete after a further 2 h. The reaction mixture was partitioned (DCM and water), the aqueous phase was extracted (DCM), the organic phases were combined, washed (water×2, then brine) then dried (Na₂SO₄) and evaporated. Flash chromatography, eluting with 100% ethyl acetate then 5% methanol/ethyl acetate, furnished an orange oil. The oil was triturated with ether to form the title compound as an amorphous orange solid (40 mg, 51%); TLC R_{f}=0.3, ethyl acetate. ¹H NMR (500 MHz, DMSO) δ 11.15 (1H, s, NH), 8.39 (1H, d, J=5.0, HarH), 8.08 (1H, s, NCH), 7.33 (1H, s, NCH), 7.09 (1H, d, J=5.0, HarH), 6.35 (1H, s, NCHCF₂), 6.18 (1H, s, OH), 6.10 (1H, q, J=6.6, OCHCH₃), 5.31 (1H, s, OH), 4.20 (1H, m, CF₂CHOH), 3.89 (1H, m, OCHCH₂OH), 3.80 (1H, m, CH₂OH), 3.65 (1H, m, CH₂OH), 1.68 (3H, d, J=6.6, CH₃) ppm.

The di-O-t-butoxycarbonyl N⁴-(1-(5-nitrothien-2-yl)ethyl)oxycarbonyl-2′,2′-difluoro-2′-deoxycytidine was synthesized as follows. 1-(5-nitrothien-2-yl)ethan-1-ol (177 mg, 1.02 mmol), 3′,5′-Di-BOC-gemcitabine (157 mg, 0.34 mmol), pyridine (0.15 mL, 1.86 mmol) and DCM (3 mL) were stirred together at 0° C. A solution of phosgene (0.25 mL, 050 mmol, 2M in toluene) was added dropwise to the reaction mixture and stirring continued for 18 h. The reaction mixture was partitioned (ethyl acetate and water), the aqueous phase was extracted (ethyl acetate), the organic phases were combined, washed (water then brine) then dried (Na₂SO₄) and evaporated. Flash chromatography, eluting with 20% and 33% ethyl acetate/hexane then 100% ethyl acetate, furnished a yellow oil (133 mg, 59%); TLC R10.8, ethyl acetate.

### EXAMPLE 4

### Tri-O-acetyl-N⁴-(1-(5-nitrothien-2-yl)ethyl)oxycarbonyl-1-β-D-arabinofuranosylcytosine

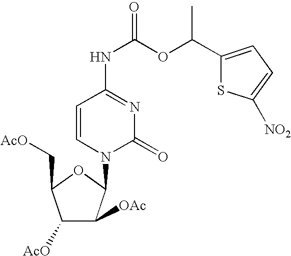

1-(5-nitrothien-2-yl)ethan-1-ol (260 mg, 1.50 mmol), triacetyl-Ara-C (406 mg, 1.00 mmol), pyridine (0.25 mL, 3.00 mmol) and DCM (2 mL) were stirred at 0° C. A solution of phosgene (0.6 mL, 1.20 mmol, 2M in toluene) was added dropwise to the reaction mixture and stirring continued for 18 h. The reaction mixture was partitioned (ethyl acetate and water), the aqueous phase was extracted (ethyl acetate), the organic phases were combined, washed (water then brine) then dried (Na₂SO₄) and evaporated. Flash chromatography, eluting with 20% and 60% ethyl acetate/hexane then 100% ethyl acetate, furnished the title compound as a yellow oil (422 mg, 74%); TLC R_{f}=0.5, ethyl acetate. ¹H NMR (270 MHz, DMSO) δ 11.03 (1H, s, NH), 8.06 (2H, s, HarH), 7.31 (1H, s, HarH), 7.06 (1H, s, NCH═CH), 6.22 (1H, s, NCHO), 6.11 (1H, bs, OCHCH₃), 5.40 (2H, s, 2×CHOH), 5.16 (1H, s, CHOH), 4.37 (2H, bs, CH₂OAc), 2.27 (9H, s, COCH₃), 1.65 (3H, d, J=6.5, OCHCH₃) ppm.

### EXAMPLE 5

### N⁴-(2-nitro-1-methylimidazol-5-yl)methoxycarbonyl-2′,2′-difluoro-2′-deoxycytidine

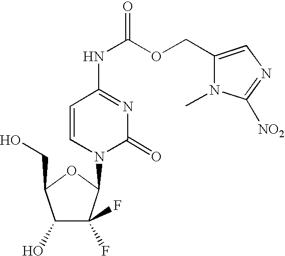

N⁴-(2-nitro-1-methylimidazol-5-yl)methoxycarbonyl-3′,5′-di-O-t-butoxycarbonyl-2′,2′-difluoro-2′-deoxycytidine (80 mg, 0.124 mmol) was dissolved in DCM (4 mL) then cooled to 0° C. TFA (1.5 mL) was added dropwise to the cooled reaction mixture and stirring continued for 4 h. Silica gel (1.0 g) was then added together with DCM (5 mL) and then the reaction mixture was evaporated. Flash chromatography, eluting with 100% ethyl acetate then 10% methanol/ethyl acetate, furnished an oil. The oil was triturated with ether to give a white solid (50 mg, 100%). NMR (500 Mhz, DMSO-d₆, δ) 11.09 (1H, s, NH), 8.36 (1H, s, NCH), 7.07 (1H, m, HarH), 6.34 (1H, s, NCHCF₂), 6.18 (1H, m, OH), 5.32 (3H, m, OCH₂ & OH), 4.20 (1H, bs, CHOH), 3.95 (3H, s, NCH₃) 3.89 (1H, m, OCHCH₂OH), 3.80 (1H, m, CH₂OH), 3.65 (1H, m, CH₂OH) ppm

N⁴-(2-nitro-1-methylimidazol-5-yl)methoxycarbonyl-3′,5′-di-O-t-butoxycarbonyl-2′,2′-difluoro-2′-deoxycytidine was synthesized as follows: 5-Hydroxymethyl-1-methyl-2-nitroimidazole (157 mg, 1.00 mmol), 3′,5′-di-O-t-butoxycarbonyl-2′,2′-difluoro-2′-deoxycytidine (200 mg, 0.43 mmol), pyridine (0.20 mL, 2.54 mmol) and DCM (3 mL) were stirred at 0° C. A solution of phosgene (0.25 mL, 0.50 mmol, 2 M in toluene) was added dropwise to the reaction mixture and stirring continued for 48 h. The reaction mixture was partitioned (ethyl acetate and water), the aqueous phase was extracted (ethyl acetate), the organic phases were combined, washed (water then brine) then dried (Na₂SO₄) and evaporated. Flash chromatography, eluting with 33% ethyl acetate/hexane then 100% ethyl acetate, furnished a yellow oil (80 mg, 29%).

### EXAMPLE 6

### N⁴-(5-nitrothien-2-yl)methoxycarbonyl-2′,2′-difluoro-2′-deoxycytidine

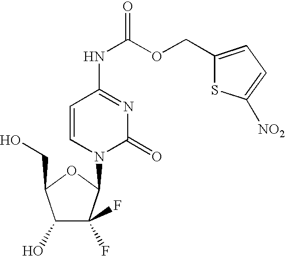

3′,5′-di-O— (t-butoxycarbonyl)-N⁴-(5-nitrothien-2-yl)methoxycarbonyl-2′,2′-difluoro-2′-deoxycytidine (50 mg, 0.08 mmol) was dissolved in DCM (3 mL) and cooled to 0° C. TFA (1 mL) was added slowly and the solution stirred at 0° C. for 2 h, and then stored at −18° C. for 12 h. A further 1 mL of TFA was then added and the solution stirred for 6 h at 0° C. Silica gel (0.5 g) was added and the solution evaporated to dryness. The preabsorbed sample was purified by flash chromatography, eluting with 10% methanol/ethyl acetate to give the title compound (6 mg, 17%); mpt=144-148° C. TLC R^{f}=0.34, ethyl acetate. LC-RT 3.9 min (TFA20-50%); MS m/z 448 (M⁺)/402/354/263/189/159/143. ¹H NMR (500 MHz, DMSO) δ 11.20 (1H, s, NH), 8.29 (1H, d, J=3.6 Hz, HarH), 8.07 (1H, s, NCH), 7.33 (1H, s, NCH), 7.10 (1H, d, J=3.6 Hz, HarH), 6.40 (1H, s, OH), 6.13 (1H, s, NCHCF₂), 5.44 (2H, s, OCH₂), 4.21 (1H, m, CHOH), 3.88 (1H, m, OCHCH₂OH), 3.80 (1H, m, CH₂OH), 3.66 (1H, m, CH₂OH) ppm. 3′,5′-di-O— (t-butoxycarbonyl)-N⁴-(5-nitrothien-2-yl)methoxycarbonyl-2′,2′-difluoro-2′-deoxycytidine was prepared as follows: 3′,5′-di-O-t-butoxycarbonyl-2′,2′-difluoro-2′-deoxycytidine (200 mg, 0.43 mmol) was dissolved in DCM (3 ml) together with pyridine (0.2 ml) and 5-nitrothien-2-ylmethanol (159 mg, 1 mmol) and the solution cooled to 0° C. Phosgene solution (0.25 ml of a 2M solution in toluene, 0.5 mmol) was slowly added at 0° C. and the solution refrigerated for 48 h. The solution was partitioned (ethyl acetate and brine), dried and evaporated. The residue was purified by flash chromatography, eluting with 25% ethyl acetate/hexane, 50% ethyl acetate/hexane and then ethyl acetate to give 50 mg (18%) of the title compound as a colourless oil. TLC R_{f}=0.1, 33% ethyl acetate/hexane. LC-RT 3.89 min (TFA50-100%)

### EXAMPLE 7

### N⁴-(5-nitro-1-methylimidazol-2-yl)methoxycarbonyl-1-β-D-arabinofuranosylcytosine

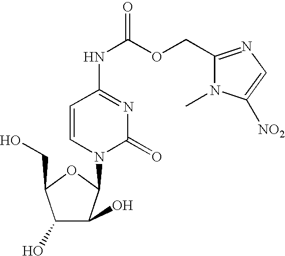

Cytarabine (243 mg, 1.0 mmol), 2-Chlorocarbonyloxymethyl-1-methyl-5-nitroimidazole (439 mg, 2.0 mmol), sodium bicarbonate (336 mg, 4.0 mmol) and DMA (10 mL) were stirred for 7 days. The suspension was filtered and the filtrate concentrated in vacuo. The resultant oil was triturated with DCM; the suspension was filtered, and the solid was washed with DCM then dried at the pump. The solid was dissolved in methanol and then adsorbed on to flash silica. Column chromatography, eluting with 33% then 50% methanol/DCM, furnished an off-white wax (53 mg, 12%); TLC R_{f}=0.3, 10% methanol/ethyl acetate. NMR (500 Mhz, DMSO-d6, δ) 7.98 (1H, s, HarH), 7.35 (1H, bs, HarH), 5.99 (1H, s, NCHO), 5.86 (1H, bs, HarH), 5.65 (1H, bs, OH), 5.47 (1H, bs, OH), 5.26 (2H, m, HarCH₂O), 5.05 (1H, s, OH), 4.08 (1H, bs, CHOH), 3.95 (3H, s, NCH₃), 3.90 (1H, bs, OCHCH₂OH), 3.73 (1H, bs, CHOH), 3.62 (2H, m, CH₂OH) ppm.

The 2-chlorocarbonyloxymethyl-1-methyl-5-nitroimidazole used in the above synthesis was prepared as follows: 2-Hydroxymethyl-1-methyl-5-nitroimidazole (314 mg, 2.0 mmol) in THF (10 mL) was added to phosgene (4 mL, 8.0 mmol) and THF (15 mL) at 0° C. The reaction was stirred for 16 h then the solvent was removed in vacuo. The crude chloroformate was used without further purification.

2-Hydroxymethyl-1-methyl-5-nitroimidazole was prepared as follows: Methanolic ammonia (3.6 mL, 25 mmol) was added to a suspension of ronidazole (5 g, 25 mmol) and methanol (25 mL). Potassium carbonate (1.75 g, 12.5 mmol) was added and the reaction mixture heated to 50° C. for 18 h. The solution was cooled to ambient temperature then partitioned (ethyl acetate and brine), the aqueous phase was extracted (ethyl acetate); the organic phases were combined, washed (water then brine) then dried in vacuo. The desired product was obtained as an orange solid (2.3 g, 59%) and was used without further purification.

### EXAMPLE 8

### N⁶-(5-nitrothien-2-yl)methoxycarbonyl-9-β-D-arabinofuranosyladenine

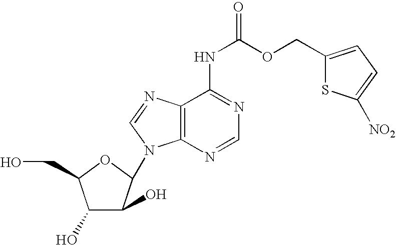

Vidarabine (267 mg, 1.0 mmol), chloroformate EE (332 mg, 1.5 mmol), sodium bicarbonate (252 mg, 3.0 mmol) and DMA (5 mL) were stirred for 48 h. The suspension was filtered and the filtrate concentrated in vacuo. The residue was dissolved in methanol and adsorbed onto flash silica in vacuo. Flash chromatography, eluting with ethyl acetate then 10% methanol/ethyl acetate, furnished a brown oil. The oil was triturated with DCM, the suspension was filtered and the filtrate concentrated under reduced pressure. Further chromatography, eluting with DCM then 10% methanol/DCM, furnished an orange oil. Methanol (3 mL) was added to the oil and after 30 min the resultant suspension was filtered and the solid was washed with ice cold methanol. The title compound was isolated as a white solid (62 mg, 14%); mpt=152-154° C.; TLC R_{f}=0.4, 10% methanol/ethyl acetate. ¹H NMR (270 MHz, DMSO) δ 8.07 (1H, s, HarH), 7.32 (1H, s, HarH), 6.88 (1H, s, HarH), 6.57 (1H, s, HarH), 5.84 (1H, s, NCHO), 5.59 (2H, s, 2×OH), 5.49 (2H, s, HarCH₂O), 4.87 (1H, s, OH), 4.28 (1H, bs, CHOH), 3.97 (1H, bs, OCHCH₂OH), 3.60-3.37 (3H, m, CHOH, CH₂OH) ppm.

### EXAMPLE 9

### 5′-Deoxy-2′,3′-di-O-acetyl-5-fluoro-N⁴-((5-nitrothien-2-yl)methoxycarbonyl)cytidine

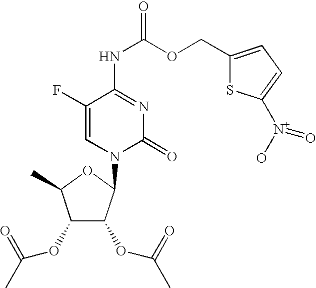

Capecitabine (5′-deoxy-5-fluoro-N⁴-(pentyloxycarbonyl)cytidine, 4.5 g, 12.5 mmol) was suspended in methanol (250 mL) and DMF (10 mL) together with potassium carbonate (8.8 g, 63.77 mmol) and the solution heated under reflux for 24 h. The solution was then cooled and evaporated to dryness (below 45° C.). The residue was redissolved in hot methanol, filtered and washed with hot methanol. The filtrate was preabsorbed onto silica gel and purified by flash chromatography, eluting with 50% methanol/ethyl acetate to give 3.0 g (90%) of 1-[3,4-dihydroxy-5-methyl-tetrahydrofuran-2-yl]-4-amino-1H-pyrimidin-2-one. This material was suspended in chloroform (125 mL) and the solution heated to 50° C. Acetic acid (2 mL, 34 mmol) was added and after 10 minutes at 50° C., acetyl chloride (20 mL, 206 mmol) was added. The suspension was stirred at 50° C. for 7 h and then at 20° C. for 72 h. Ether (100 mL) was added and the solid filtered and washed with ether to give 4.0 g (90%) 1-[3,4-Diacetoxy-5-methyl-tetrahydrofuran-2-yl]-4-amino-1H-pyrimidin-2-one hydrochloride. This material (2.07 g, 5.7 mmol) together with 5-nitrothien-2-ylmethanol (1.47 g, 9.3 mmol), was dissolved in pyridine (1.4 mL, 17.4 mmol) and DCM (15 mL). Phosgene solution (3.6 mL of a 2M solution in toluene, 7.2 mmol) was slowly added to the above cooled (0° C.) solution, the solution was stirred at 0° C. for 2.5 h and then a further 3.6 mL of phosgene solution added and the solution stirred at 0° C. for a further 2 h and refrigerated for 18 h. The solution was partitioned (ethyl acetate and brine), the aqueous phase extracted (ethyl acetate) dried and evaporated. The residue was purified on silica, eluting with 2% methanol/DCM, to give an off-white foam (400 mg, 14%); TLC R_{f}=0.45, 2% methanol/ethyl acetate. LC-RT 4.68 min (TFA20-50%); MS m/z 201/159/143. ¹H NMR (500 MHz, DMSO) δ 11.20 (1H, bs, NH), 8.3 (1H, b, NCH═CF), 8.05 (1H, s, HarH), 7.30 (s, 1H, HarH), 5.80 (1H, s, NCHO), 5.42 (3H, m, HarCH₂OCONH, CHOAc), 5.15 (1H, s, CHOAc), 4.05 (1H, m, OCHCH₃), 3.45 (2H, m, 2×CHOAc), 2.48 (3H, s, OAc), 2.05 (3H, s, OAc), 1.37 (3H, s, OCHCH₃) ppm.

### EXAMPLE 10

### 2-fluoro-N⁶-(5-nitrothien-2-yl)methoxycarbonyl-9-O-D-arabinofuranosyladenine

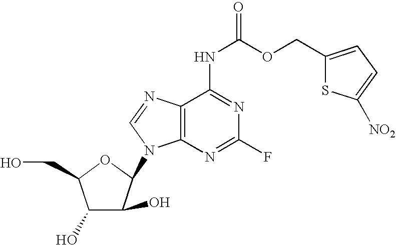

Fludarabine ((2-fluoro-9-(β-D-arabinofaranosyl)-9H-purin-6-amine), 187 mg, 0.66 mmol), chloroformate EE (443 mg, 2.00 mmol), sodium bicarbonate (168 mg, 2.00 mmol) and DMA (5 mL) were stirred for 7 days. The suspension was filtered and the filtrate concentrated in vacuo. The residue was dissolved in methanol and adsorbed onto flash silica in vacuo. Flash chromatography, eluting with ethyl acetate then 10% methanol/ethyl acetate, furnished an amber oil. Methanol (5 mL) was added to the oil and after 4 h the resultant suspension was filtered and the solid was washed with ice cold methanol. The title compound was isolated as a beige solid (112 mg, 36%); mpt=152-154° C.; TLC R_{f}=0.5, ethyl acetate. ¹H NMR (270 MHz, DMSO) δ 8.06 (1H, s, HarH), 7.30 (1H, s, HarH), 6.64 (1H, s, HarH), 5.80 (1H, s, NCHO), 5.62 (2H, s, 2×OH), 5.51 (2H, s, HarCH₂O), 4.88 (1H, s, OH), 4.36 (1H, bs, CHOH), 4.01 (1H, bs, OCHCH₂OH), 3.62-3.3.39 (3H, m, CHOH, CH₂OH) ppm.

### EXAMPLE 11

### N⁴-(1-(4-nitrophenyl)ethoxycarbonyl)-1-β-D-arabinofuranosylcytosine

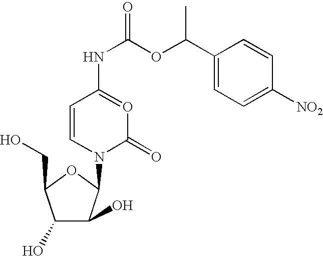

Cytarabine (735 mg, 3.0 mmol), 1-chlorocarbonyloxy-1-(4-nitrophenyl)ethane (2.29 g, 10.0 mmol), sodium bicarbonate (882 mg, 10.5 mmol) and DMA (20 mL) were stirred for 7 days. The suspension was filtered and the filtrate concentrated in vacuo. The solid was dissolved in methanol and then adsorbed on to flash silica. Column chromatography, eluting with ethyl acetate then 10% methanol/ethyl acetate, produced a yellow oil. The oil was triturated with acetonitrile; the suspension was filtered, and the solid was washed with ice-cold acetonitrile then dried at the pump. The title compound was obtained as a cream powder (292 mg, 22%); mpt=144-146° C.; TLC R_{f}=0.1, ethyl acetate. ¹H NMR (270 MHz, DMSO) δ 10.86 (1H, s, NH), 8.27 (2H, d, J=8.1, ArH), 8.04 (1H, d, J=8.1, NCH═CH), 7.68 (2H, d, J=8.1, ArH), 6.95 (1H, d, J=8.1, NCH═CH), 6.04 (1H, d, J=5.4, NCHO), 5.95 (1H, q, J=5.4, OCHCH₃), 5.49 (2H, m, 2×OH), 5.05 (1H, t, J=5.4, OH), 4.05 (1H, bs, CHOH), 3.91 (1H, bs, OCHCH₂OH), 3.83 (1H, bs, CHOH), 3.59 (2H, t, J=5.4, CH₂OH), 1.54 (3H, d, J=5.4, OCHCH₃) ppm.

1-Chlorocarbonyloxy-1-(4-nitrophenyl)ethane was synthesized as follows: 2-(4-Nitrophenyl)ethanol (1.67 g, 10.0 mmol) in THF (10 mL) was added to phosgene (5.5 mL, 11.0 mmol) and THF (30 mL) at 0° C. The reaction was stirred for 16 h then the solvent was removed in vacuo. The crude chloroformate was used without further purification.

### EXAMPLE 12

### N⁴-(5-nitrofuran-2-yl)methoxycarbonyl-1-β-D-arabinofuranosylcytosine

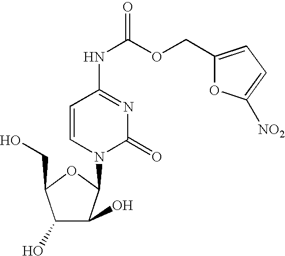

Cytarabine (812 mg, 3.31 mmol), 2-Chlorocarbonyloxymethyl-5-nitrofuran (2.05 g, 10.00 mmol), sodium bicarbonate (980 mg, 11.66 mmol) and DMA (10 mL) were stirred for 72 h. The suspension was filtered and the filtrate concentrated in vacuo to give a solid, which was dissolved in methanol and then adsorbed on to flash silica. Column chromatography, eluting with ethyl acetate then 10% methanol/ethyl acetate, produced an impure oil. Further flash chromatography, eluting with DCM then 5%, 10%, and 15% methanol/DCM, afforded an amber oil. The oil was triturated with ether; the suspension was filtered, and the solid was washed with ether then dried at the pump. The title compound was obtained as a beige powder (95 mg, 7%); mpt=105-107° C.; TLC R_{f}=0.18, 10% methanol/ethyl acetate. ¹H NMR (270 MHz, DMSO) δ 10.91 (1H, s, NH), 8.07 (2H, s, HarH), 7.70 (1H, s, HarH), 6.98 (1H, s, NCH═CH), 6.04 (1H, s, NCHO), 5.49 (2H, s, 2×OH), 5.26 (2H, s, HarCH₂O), 5.05 (1H, s, OH), 4.10 (1H, bs, CHOH), 3.91 (1H, bs, OCHCH₂OH), 3.82 (1H, bs, CHOH), 3.59 (2H, m, CH₂OH) ppm.

2-Chlorocarbonyloxymethyl-5-nitrofuran was prepared as follows: 2-Hydroxymethyl-5-nitrofuran (1.43 g, 10.0 mmol) in THF (10 mL) was added to phosgene (5.5 mL, 11.0 mmol) and THF (30 mL) at 0° C. The reaction was stirred for 16 h then the solvent was removed in vacuo. The crude chloroformate was used without farther purification.

### EXAMPLE 13

### N⁴-(5-methoxy-1,2-dimethyl-4,7-dioxoindol-3-yl)methoxycarbonyl-1-β-D-arabinofuranosylcytosine

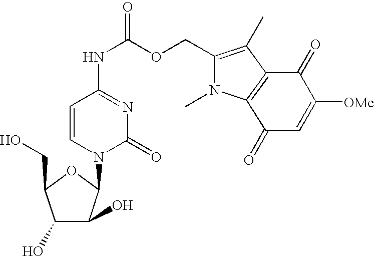

3-Hydroxymethyl-5-methoxy-1,2-dimethylindole-4,7-dione (150 mg, 0.64 mmol) was dissolved in pyridine (0.5 mL) and the solution cooled to 0° C. A solution of 4-nitrophenylchloroformate (200 mg, 1 mmol) in pyridine (0.5 mL) was then added drop-wise and the solution warmed to 20° C. over 1 h. The solution was partitioned (ethyl acetate/brine) and extracted further with ethyl acetate, dried and evaporated to dryness. The crude 4-nitrophenylcarbonate was dissolved in DMA (2 mL) together with cytarabine (500 mg, 2.05 mmol) and sodium bicarbonate (500 mg, 5.95 mmol). The solution was stirred at 20° C. for 24 h and then evaporated to dryness at 30° C., dissolved in methanol, filtered and absorbed onto silica gel (2.5 g). The material was then purified by flash chromatography, eluting with ethyl acetate followed by 10% methanol/ethyl acetate to afford the title compound as an orange solid (20 mg, 10%); mpt=>250° C. (dec.). TLC R_{f}=0.2, (10% methanol/ethyl acetate). LC-RT 2.16 min (TFA20-50%); MS m/z 235/220/151. ¹H NMR (500 MHz, DMSO) δ 10.55 (1H, s, NH), 8.05 (1H, s, NCH═CH), 7.00 (1H, s, NCH═CH), 6.05 (1H, s, NCHO), 5.79 (1H, s, HarH), 5.54 (2H, s, 2×OH), 5.25 (s, 2H, HarCH₂OCONH), 5.10 (1H, s, OH), 4.05 (1H, bs, CHOH), 3.90 (s, 3H, HarOCH₃), 3.91 (1H, bs, OCHCH₂OH), 3.85 (s, 3H, HarNCH₃), 3.84 (1H, bs, CHOH), 3.61 (2H, m, CH₂OH), 2.28 (3H, s, HarCH₃) ppm.

### EXAMPLE 14

### 5′-Deoxy-5-fluoro-N⁴-((5-nitrothien-2-yl)methoxycarbonyl)cytidine

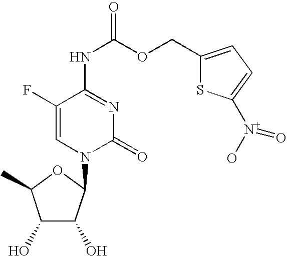

5′-Deoxy-2′,3′-di-O-acetyl-5-fluoro-N⁴-((5-nitrothien-2-yl)methoxycarbonyl)cytidine (200 mg, 0.4 mmol) was dissolved in MeOH (8 ml) and cooled to −10° C. NaOH (64 mg, 1.6 mmol) in water (1 ml) was added drop-wise over 30 min. and the solution stirred at −10° C. for 1 h and then at 4° C. for 1 h. The solution was neutralised with 3M HCl and evaporated to dryness (high vac, below 40° C.), re-dissolved in acetone, and purified on silica (eluting with 50% ethyl acetate/methanol) to five a pale yellow foam (25 mg, 14.5%). LCMS rt=3.926° (TFA20-50) m/e=430 (M⁺)/343/314/271/156/143.

1. A compound of formula (1), or a pharmaceutically acceptable salt thereof, 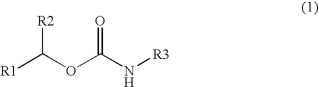
wherein:
R1 is a substituted aryl or heteroaryl group bearing at least one nitro or azido group or is an optionally substituted benzoquinone, optionally substituted naphthoquinone or optionally substituted fused heterocycloquinone;
R2 is H, optionally substituted alkyl, optionally substituted alkenyl, optionally substituted alkynyl, optionally substituted cycloalkyl, optionally substituted heterocycloalkyl, aryl or heteroaryl; and
R3 is selected such that R3NH₂ represents a cytotoxic nucleoside analogue or an ester or phosphate ester prodrug of a cytotoxic nucleoside analogue, with the proviso that if R1 is an aryl group then R2 is not H.

2. A compound according to claim 1, wherein:
the alkyl, alkenyl and alkynyl groups are unsubstituted or substituted with 1, 2 or 3 unsubstituted substituents selected from halogen, amino, mono(C₁-C₄ alkyl)amino, di(C₁-C₄ alkyl)amino, hydroxy, C₁-C₄ alkoxy, C₁-C₄ alkylthio, (C₁-C₄ alkyl)sulphonyl groups, aryl, heteroaryl, heterocycloalkyl, acylamino, (C₁-C₄)alkoxycarbonylamino, (C₁-C₄)alkanoyl, acyloxy, carboxy, sulphate or phosphate groups;
the aryl and heteroaryl groups are unsubstituted or substituted with 1, 2 or 3 unsubstituted substituents selected from halogen, C₁-C₆ alkyl, hydroxy, nitro, azido, cyano, amino, C₁-C₄ alkylamino, di(C₁-C₄)alkylamino, acylamino, C₁-C₄ alkoxycarbonylamino, C₁-C₄ alkanoyl, acyloxy, carboxy, aminocarbonyl, C₁-C₄ alkylaminocarbonyl, di(C₁-C₄)alkylaminocarbonyl, (C₁-C₄)alkylthio, C₁-C₄ alkoxy, C₁-C₄ haloalkyl, and C₁-C₄ haloalkoxy;
the heterocycloalkyl ring is unsubstituted or substituted with 1, 2 or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, C₁-C₄ alkylamino, di(C₁-C₄)alkylamino, carboxy, (C₁-C₄)alkoxycarbonyl, aminocarbonyl, (C₁-C₄)alkylaminocarbonyl, di(C₁-C₄)alkylaminocarbonyl, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate;
cycloalkyl group is unsubstituted or substituted with 1, 2, or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, C₁-C₄ alkylamino, di(C₁-C₄)alkylamino, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate;
the benzoquinone group is unsubstituted or substituted by 1, 2 or 3 unsubstituted substituents selected from C₁-C₆ alkyl, C₁-C₄ haloalkyl, C₁-C haloalkoxy, halogen, hydroxy, C₁-C₄ alkoxy, C₁-C₄ alkylthio, amino, C₁-C₄ alkylamino, di(C₁-C₄)alkylamino, heterocycloalkyl, cycloalkyl, aryl or heteroaryl; and
the naphthoquinone or fused heterocycloquinone group is unsubstituted or substituted by 1, 2, 3 or 4 unsubstituted substituents selected from C₁-C₆ alkyl, C₁-C₄ haloalkyl, C₁-C haloalkoxy, halogen, hydroxy, C₁-C₄ alkoxy, C₁-C₄ alkylthio, amino, C₁-C₄ alkylamino, di(C₁-C₄)alkylamino, heterocycloalkyl, cycloalkyl, aryl or heteroaryl.

3. Compound according to claim 1 wherein R1 is either:
(a) a phenyl or 5- to 6-membered heteroaryl group carrying one substituent selected from a nitro or azido group and 0, 1 or 2 further unsubstituted substituents selected from C₁-C₂ alkyl, C₁-C₂ haloalkyl, C₁-C₂ alkoxy and C₁-C₂ haloalkoxy substituents; or
(b) a benzoquinone, naphthoquinone or a fused heterocycloquinone group wherein a benzoquinone group is fused to a 5- to 6-membered heteroaryl group, which is unsubstituted or substituted by 1, 2, or 3 unsubstitued substituents selected from C₁-C₂ alkyl, C₁-C₂ haloalkyl, C₁-C₂ haloalkoxy, C₁-C₂ alkoxy and C₁-C₂ alkylthio groups.

4. Compound according to claim 1 wherein R2 is H or an unsubstituted C₁-C₄ alkyl group.

5. A compound according to claim 1 wherein R3 is selected from a group of formula (2), (3) or (4): 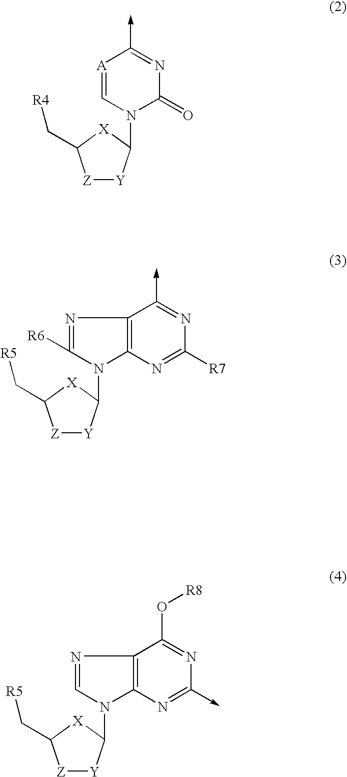
in which:
A is N, CF or CH;
X is O or S;
Y is CH₂, CHOH, CHO(CO)alkyl, CHF, CF₂, CHCN, C═CH₂, or C═CHF;
Z is CHOH, CR9′OH, CHOP(O)(OH)₂, CHOC(O)alkyl or 0;
R4 is H, OH, OP(O)(OH)₂ or OC(O)alkyl;
R5 is OH, OP(O)(OH)₂ or OC(O)alkyl;
R6 is H, Cl or F;
R7 is H, Cl or F;
R8 is H or alkyl; and
R9′ is alkyl, alkenyl or alkynyl, with the proviso that R3NH₂ does not represent the natural nucleosides cytidine, 2′-deoxycytidine, adenosine, 2′-deoxyadenosine, guanosine, 2′deoxyguanosine or a cytidine, 2′-deoxycytidine, adenosine, 2′-deoxyadenosine guanosine, 2′deoxyguanosine prodrug, and, further, when R4 is H then A is CF, X is O and Y is CHOH or CHO(CO)alkyl.

6. Compound according to claim 5 wherein A is CH or CF.

7. Compound according to claim 5 wherein X is O.

8. Compound according to claim 5 wherein Y is an unsubstituted CH₂, CHOH, CHO(CO)—C₁-C₄ alkyl, CHF or CF₂ group.

9. Compound according to claim 5 wherein Z is an unsubstituted CHOH or CHOC(O)—C₁-C₄ alkyl group.

10. Compound according to claim 5 wherein R4 is H or an unsubstituted OH or OC(O)—C₁-C₄ alkyl group.

11. Compound according to claim 5 wherein R5 is H or an unsubstituted OH or OC(O)—C₁-C₄ alkyl group.

12. Compound according to claim 5 wherein R6 is H.

13. Compound according to claim 5 wherein R7 is H or F.

14. Compound according to claim 5 wherein R8 is H or an unsubstituted C₁-C₄ alkyl group.

15. Compound according to claim 5 wherein R9′ is an unsubstituted C₂-C₄ alkynyl group.

16. Compound according to claim 5 wherein R3 is a group of formula (2) or formula (3).

17. Compound according to claim 1 wherein R1 is a substituted phenyl or 5-membered heteroaryl group bearing at least one nitro or azido substituent.

18. Compound according to claim 5 wherein Y is an unsubstituted CH₂, CHOH, CHO(CO)—C₁-C₆ alkyl or CHF group.

19. Compound according to claim 5 wherein when R3 is a group of formula (2), either:
(a) A is CH; or
(b) R4 is an unsubstituted OH, OP(O)(OH)₂ or OC(O)—C₁-C₆ alkyl group.

20. Compound according to claim 1 wherein R3 is selected such that R3NH₂ represents gemcitabine, cytarabine (1-β-D-arabinofuranosylcytosine), fludarabine phosphate (2-fluoro-9-(5-O-phosphono-β-D-arabinofuranosyl)-9H-purin-6-amine), fludarabine(2-fluoro-9-(-β-D-arabinofuranosyl)-9H-purin-6-amine), cladribine (2-chloro-2′-deoxy-β-D-adenosine), troxacitabine (2′deoxy-3′oxacytidine), 5-azacytidine, decitabine (5-aza-2′-deoxycytidine), tezacitabine (E-2′deoxy-2-fluoromethylene)cytidine), DMDC (1-(2-deoxy-2-methylene-β-D-erythro-pentofuranosyl)cytosine), clofarabine (2-chloro-2′-fluoro-deoxy-9-β-D-arabinofuranosyladenine), fazarabine (1-β-D-arabinofuranosyl-5-azacytosine), vidarabine (9-β-D-arabinosyladenine), CNDAC (1-(2-C-cyano-2-deoxy-β-D-arabino-pentofuranosyl)-cytosine), OSI-7836 (4′-thio-aracytidine), 4-thio-FAC (1-(2-deoxy-2-fluoro-4-thio-β-D-arabinofuranosyl)cytosine), TAS-1061 ((3-C-ethynyl-β-D-ribo-pentofuranosyl)cytosine), ara-G (9-β-D-arabinofuranosyl guanine), nelarabine (2-amino-9-β-D-arabinofuranosyl-6-methoxy-9H-purine), 5′-deoxy-5-fluorocytidine, 2′,3′-di-O-acetyl-5′-deoxy-5-fluorocytidine or 2′,3′,5′-tri-O-acetyl-cytarabine.

21. Compound according to claim 1 which is N⁴-(1-(5-nitrothien-2-yl)ethyl)oxycarbonyl-1-β-D-arabinofuranosylcytosine;
N⁴-(5-nitrothien-2-yl)methoxycarbonyl-1-β-D-arabinofuranosylcytosine;
N⁴-(1-(5-nitrothien-2-yl)ethyl)oxycarbonyl-2′,2′-difluoro-2′-deoxycytidine;
Tri-O-acetyl-N⁴-(1-(5-nitrothien-2-yl)ethyl)oxycarbonyl-1-β-D-arabinofuranosylcytosine;
N⁴-(2-nitro-1-methylimidazol-5-yl)methoxycarbonyl-2′,2′-difluoro-2′-deoxycytidine;
N⁴-(5-nitrothien-2-yl)methoxycarbonyl-2′,2′-difluoro-2′-deoxycytidine;
N⁴-(5-nitro-1-methylimidazol-2-yl)methoxycarbonyl-1-β-D-arabinofuranosylcytosine;
N⁶-(5-nitrothien-2-yl)methoxycarbonyl-9-β-D-arabinofuranosyladenine;
5′-Deoxy-2′,3′-di-O-acetyl-5-fluoro-N⁴-((5-nitrothien-2-yl)methoxycarbonyl)cytidine;
2-fluoro-N⁶-(5-nitrothien-2-yl)methoxycarbonyl-9-β-D-arabinofuranosyladenine;
N⁴-(1-(4-nitrophenyl)ethoxycarbonyl)-1-β-D-arabinofuranosylcytosine;
N⁴-(5-nitrofuran-2-yl)methoxycarbonyl-1-β-D-arabinofuranosylcytosine;
N⁴-(5-methoxy-1,2-dimethyl-4,7-dioxoindol-3-yl)methoxycarbonyl-1-β-D-arabinofuranosylcytosine, or 5′-Deoxy-5-fluoro-N⁴-((5-nitrothien-2-yl)methoxycarbonyl)cytidine, or a pharmaceutically acceptable salt thereof.

22. A pharmaceutical composition comprising a compound according to claim 1, or a pharmaceutically acceptable salt thereof, and a pharmaceutically acceptable carrier or diluent.

23. A method of ameliorating or reducing the incidence of a proliferative disorder in a patient, which method comprises administering to said patient an effective amount of a compound as defined in claim 1, or a pharmaceutically acceptable salt thereof.

24. (canceled)

25. A method according to claim 23, wherein the proliferative disorder is cancer, rheumatoid arthritis, psoriatic lesions, diabetic retinopathy or wet age-related macular degeneration.

26. A method according to claim 23, wherein the proliferative disorder is a hypoxic disorder.

27. A method according to claim 23, wherein the proliferative disorder is a solid tumour or leukaemia.

28. (canceled)

29. A method of ameliorating or reducing the incidence of a proliferative disorder in a patient, which method comprises administering to said patient an effective amount of
(a) a compound as defined in claim 1, or a pharmaceutically acceptable salt thereof; and
(b) a reductase, an anti-body reductase conjugate, a macromolecule-reductase conjugate or DNA encoding a reductase gene.

30. (canceled)
